(19) United States
(12) Patent Application Publication
(10) Pub. No.: US 2012/0245699 A1
(43) Pub. Date: Sep. 27, 2012

(54) JOINT ARTHROPLASTY DEVICES

(75) Inventors: Philipp LANG, Lexington, MA (US); Albert G. BURDULIS, JR., San Francisco, CA (US); Wolfgang FITZ, Sherborn, MA (US); Daniel STEINES, Palo Alto, CA (US)

(73) Assignee: ConforMIS, Inc., Burlington, MA (US)

(21) Appl. No.: 13/421,554

(22) Filed: Mar. 15, 2012

Related U.S. Application Data
(63) Continuation of application No. 11/537,318, filed on Sep. 29, 2006.
Application No. 11/537,318 is a continuation-in-part of application No. 10/681,749, filed on Oct. 7, 2003, now Pat. No. 7,799,077.
Application No. 10/681,749 is a continuation-in-part of application No. 10/997,407, filed on Nov. 24, 2004.
Application No. 10/997,407 is a continuation-in-part of application No. 10/681,750, filed on Oct. 7, 2003.
Application No. 10/997,407 is a continuation-in-part of application No. 10/752,438, filed on Jan. 5, 2004.
Application No. 10/752,438 is a continuation-in-part of application No. 10/724,010, filed on Nov. 25, 2003, now Pat. No. 7,618,451.
Application No. 10/724,010 is a continuation-in-part of application No. 10/305,652, filed on Nov. 27, 2002, now Pat. No. 7,468,075.
Application No. 10/305,652 is a continuation-in-part of application No. 10/160,667, filed on May 28, 2002.
(60) Provisional application No. 60/416,601, filed on Oct. 7, 2002.
(60) Provisional application No. 60/467,686, filed on May 2, 2003.
(60) Provisional application No. 60/293,488, filed on May 25, 2001.
(60) Provisional application No. 60/363,527, filed on Mar. 12, 2002.
(60) Provisional application No. 60/380,692, filed on May 14, 2002.
(60) Provisional application No. 60/380,695, filed on May 14, 2002.
(60) Provisional application No. 60/722,171, filed on Sep. 30, 2005.

(51) Int. Cl. A61F 2/38; A61F 2/30

(52) U.S. Cl. 623/20.3; 623/18.11; 623/20.33

(57) ABSTRACT

A mobile bearing implant includes a first component. The first component includes a bone facing surface for engaging one of a substantially uncut articular cartilage surface and a substantially uncut subchondral bone surface. The bone facing surface substantially matches the one of the articular cartilage surface and the subchondral bone surface. The mobile bearing implant further includes an external surface. A bearing component has a first surface for slidingly engaging the external surface of the first component, and a second surface for engaging at least one of a second component, bone, and cartilage.

### CROSS REFERENCE TO RELATED APPLICATIONS

This application is a continuation of U.S. patent application Ser. No. 11/537,318, entitled “Joint Arthroplasty Devices,” filed Sep. 29, 2006, which in turn claims the benefit of U.S. provisional application 60/722,171, entitled “Patient Selectable Knee Joint Arthroplasty Devices,” filed Sep. 30, 2005.

U.S. patent application Ser. No. 11/537,318 also is a continuation-in-part of U.S. patent application Ser. No. 10/997,407, entitled “Patient Selectable Knee Joint Arthroplasty Devices,” filed Nov. 24, 2004, which in turn is a continuation-in-part of U.S. patent application Ser. No. 10/752,438, entitled “Patient Selectable Knee Joint Arthroplasty Devices.” filed Jan. 5, 2004, which in turn is a continuation-in-part of U.S. patent application Ser. No. 10/724,010, entitled “Patient Selectable Joint Arthroplasty Devices and Surgical Tools Facilitating Increased Accuracy, Speed and Simplicity in Performing Total and Partial Joint Arthroplasty,” filed Nov. 25, 2003, which in turn is a continuation-in-part of U.S. patent application Ser. No. 10/305,652 entitled “Methods and Compositions for Articular Repair,” filed Nov. 27, 2002, which in turn is a continuation-in-part of U.S. patent application Ser. No. 10/160,667, entitled “Methods and Compositions for Articular Resurfacing,” filed May 28, 2002, which in turn claims the benefit of U.S. provisional patent application 60/293,488 entitled “Methods To Improve Cartilage Repair Systems,” filed May 25, 2001, U.S. provisional patent application 60/363,527, entitled “Novel Devices For Cartilage Repair,” filed Mar. 12, 2002, U.S. patent application 60/380,695, entitled “Methods And Compositions for Cartilage Repair,” filed May 14, 2002 and U.S. patent application 60/380,692, entitled “Methods for Joint Repair,” filed May 14, 2002.

U.S. patent application Ser. No. 10/997,407 is also a continuation-in-part of U.S. application Ser. No. 10/681,750, filed Oct. 7, 2003, entitled “Minimally Invasive Joint Implant with 3-Dimensional Geometry Matching the Articular Surfaces,” which in turn claims the benefit of U.S. provisional patent application 60/467,686 filed May 2, 2003 entitled “Joint Implants” and U.S. provisional patent application 60/416,601, entitled “Minimally Invasive Joint Implant with 3-Dimensional Geometry Matching the Articular Surfaces,” filed on Oct. 7, 2002.

U.S. patent application Ser. No. 11/537,318 is also a continuation-in-part of U.S. application Ser. No. 10/681,749, filed Oct. 7, 2003, entitled “Minimally Invasive Joint Implant with 3-Dimensional Geometry Matching the Articular Surfaces,”

Each of the above-described applications is incorporated herein, in their entireties, by reference.

### TECHNICAL FIELD

The present invention relates to orthopedic implants and systems, such as joint implants, interpositional implants, and mobile bearing implants.

### BACKGROUND ART

A conventional prosthetic joint implant may include single or multiple components. For example, a joint implant often referred to as a mobile bearing implant may include a bearing component that is interposed between first and second components. The bearing component extends the range of movements that can be accommodated, such as sliding and rotational movement.

Implantation of these prosthetic devices is usually associated with loss of underlying tissue and bone and, with some devices, serious long-term complications associated with the loss of significant amount of tissue and bone can include infection, osteolysis and also loosening of the implant. Such joint arthroplasties can be highly invasive and require surgical resection of the entire, or a majority of the, articular surface of one or more bones involved in the repair. Typically with these procedures, the marrow space is fairly extensively reamed in order to fit the stem of the prosthesis within the bone. Reaming results in a loss of the patient's bone stock and over time subsequent osteolysis will frequently lead to loosening of the prosthesis. Further, the area where the implant and the bone mate degrades over time requiring the prosthesis to eventually be replaced. Since the patient's bone stock is limited, the number of possible replacement surgeries is also limited for joint arthroplasty. In short, over the course of 15 to 20 years, and in some cases even shorter time periods, the patient can run out of therapeutic options ultimately resulting in a painful, non-functional joint.

Another concern with prosthetic joint implants, such as a mobile bearing implant, is to ensure full range of appropriate motion. This must be balanced with the risk of dislocation of the device.

### SUMMARY OF THE INVENTION

In accordance with a first embodiment of the invention, a mobile bearing implant includes a first component. The first component includes a bone facing surface for engaging one of a substantially uncut articular cartilage surface and a substantially uncut subchondral bone surface. The bone facing surface substantially matches the one of the articular cartilage surface and the subchondral bone surface. The mobile bearing implant further includes an external surface. A bearing component has a first surface for slidingly engaging the external surface of the first component, and a second surface for engaging at least one of a second component, bone, and cartilage.

In accordance with related embodiments of the invention, the bone facing surface of the first component may substantially match one of a substantially uncut articular cartilage surface of a tibia and a substantially uncut subchondral bone surface of a tibia, and the second surface of the bearing component engages a femoral implant component.

In further related embodiments of the invention, the external surface of the first component may be curved. The external surface may include a plurality of curved surfaces with varying radii. The external surface may be flat along at least one axis.

In yet further related embodiments of the invention, the external surface of the first component may include a slot, and the first surface of the bearing component includes an anchor. The anchor slidingly engages the slot so as to direct movement of the bearing component along the external surface of the first component. The slot may be curved. The slot may include a plurality of curvatures with varying radii. The slot may be sloped.

In still a further embodiment of the invention, the first component may include at least one stop for limiting motion of the bearing component, the stop including a curved surface for contacting the bearing component.

In accordance with another embodiment of the invention, a mobile bearing implant includes: a first component having a bone facing surface for engaging at least one of bone and cartilage; and an external surface. A bearing component has a first surface for slidingly engaging the external surface of the first component, and a second surface for engaging at least one of a second component, bone, and cartilage. The external surface includes at least one of a concavity and a convexity.

In accordance with related embodiments of the invention, the bone facing surface of the first component may engage a tibial articular surface, and the second surface of the bearing component engages a femoral implant component. The external surface of the first component may include a plurality of curved surfaces with varying radii. The external surface may be flat along at least one axis.

In accordance with further related embodiments of the invention, the external surface of the first component may includes a slot, and the first surface of the bearing component includes an anchor. The anchor slidingly engages the slot so as to direct movement of the bearing component along the external surface of the first component. The slot may be curved, or sloped. The slot may include a plurality of curvatures with varying radii.

In accordance with still further related embodiments of the invention, the first component may include at least one stop for limiting motion of the bearing component, the stop including a curved surface for contacting the bearing component.

In accordance with another embodiment of the invention, a mobile bearing implant includes a first component including: a bone facing surface for engaging one of bone and cartilage; and an external surface. The external surface includes a slot. A bearing component has a first surface for slidingly engaging the external surface of the first component, and a second surface for engaging at least one of a second component, bone, and cartilage. The first surface of the bearing component includes an anchor. The anchor slidingly engages the slot so as to direct movement of the bearing component along the external surface of the first component.

In accordance with related embodiments of the invention, the bone facing surface of the first component may engage a tibial articular surface, and the second surface of the bearing component engages a femoral implant component. The slot may be curved. The slot may include a plurality of curvatures with varying radii. The slot may be sloped.

In accordance with another embodiment of the invention, a mobile bearing implant includes: a first component having a bone facing surface for engaging one of bone and cartilage; and an external surface. A bearing component has a first surface and a second surface, the first surface for slidingly engaging the external surface of the first component, and the second surface for engaging at least one of a second component, bone, and cartilage. At least one of the first surface and the second surface includes a curved surface in one dimension, the curved surface having a plurality of radii.

In accordance with another embodiment of the invention, a mobile bearing implant includes: a first component having a bone facing surface for engaging one of bone and cartilage; and an external surface. A bearing component has a first surface and a second surface, the first surface for slidingly engaging the external surface of the first component, and the second surface for engaging at least one of a second component, bone, and cartilage. The first component has an outer perimeter of varying radii.

In accordance with related embodiments of the invention, the outer perimeter of the first component may be kidney shaped. The outer perimeter of the first component may be asymmetric. The first component may have a larger, or smaller, outer perimeter than the bearing component.

In accordance with embodiments related to the above-described embodiments, the first component may be fixedly anchored into an articular surface using one or more fins, keels, and/or pegs. The fins, keels and/or pegs may have various orientations and lengths. For example, the fins and/or pegs may be perpendicular to each other. The bone facing surface of the first component may sit on top of the natural surface of the subchondral bone or articular cartilage, with only the anchoring mechanism protruding into the bone.

In accordance with further embodiments related to the above-described embodiments, the first component may include one or more stops, that restrict motion of one or more components of the mobile bearing. The stop(s) may restrict motion in one or more dimensions. The stop(s) may be oriented in various directions. Each stop may include curved and/or straight portions For example, a stop may have a constant or variable radius.

The joint implants described herein may be implemented for the knee, hip, ankle, shoulder, elbow, wrist, and hand. The various joint implants described herein may be used, without limitation, in conjunction with knee implants, including a unicompartmental arthroplasty, medial or lateral; a bicompartmental arthroplasty that covers portions or all of one femoral condyle, medial or lateral, and the trochlea, and a total knee arthroplasty system. In a total knee arthroplasty system, the intercondylar region can be preserved by using a medial and a lateral tibial device in combination. Both devices may be a fixed, non-mobile bearing, both can be a mobile bearing, or one can be a fixed, non-mobile bearing, while the other is a mobile bearing.

### BRIEF DESCRIPTION OF THE DRAWINGS

The foregoing features of the invention will be more readily understood by reference to the following detailed description, taken with reference to the accompanying drawings, in which:

FIG. 1A is a block diagram of a method for assessing a joint in need of repair according to the invention wherein the existing joint surface is unaltered, or substantially unaltered, prior to receiving the selected implant. FIG. 1B is a block diagram of a method for assessing a joint in need of repair according to the invention wherein the existing joint surface is unaltered, or substantially unaltered, prior to designing an implant suitable to achieve the repair. FIG. 1C is a block diagram of a method for developing an implant and using the implant in a patient.

FIG. 2A is a perspective view of a joint implant of the invention suitable for implantation at the tibial plateau of the knee joint. FIG. 2B is a top view of the implant of FIG. 2A. FIG. 2C is a cross-sectional view of the implant of FIG. 2B along the lines C-C shown in FIG. 2B. FIG. 2D is a cross-sectional view along the lines D-D shown in FIG. 2B. FIG. 2E is a cross-sectional view along the lines E-E shown in FIG. 2B. FIG. 2F is a side view of the implant of FIG. 2A. FIG. 2G is a cross-sectional view of the implant of FIG. 2A shown implanted taken along a plane parallel to the sagittal plane. FIG. 2H is a cross-sectional view of the implant of FIG. 2A shown implanted taken along a plane parallel to the coronal plane. FIG. 21 is a cross-sectional view of the implant of FIG. 2A shown implanted taken along a plane parallel to the axial plane. FIG. 2J shows a slightly larger implant that extends closer to the bone medially (towards the edge of the tibial plateau) and anteriorly and posteriorly. FIG. 2K is a side view of an alternate embodiment of the joint implant of FIG. 2A showing an anchor in the form of a keel. FIG. 2L is a bottom view of an alternate embodiment of the joint implant of FIG. 2A showing an anchor. FIG. 2M shows an anchor in the form of a cross-member. FIG. 2N-O are alternative embodiments of the implant showing the lower surface have a trough for receiving a cross-bar. FIG. 2P illustrates a variety of cross-bars. FIGS. 2Q-R illustrate the device implanted within a knee joint. FIG. 2S(1-9) illustrate another implant suitable for the tibial plateau further having a chamfer cut along one edge. FIG. 2T(1-8) illustrate an alternate embodiment of the tibial implant wherein the surface of the joint is altered to create a flat or angled surface for the implant to mate with.

FIGS. 3A and B are perspective views of a joint implant suitable for use on a condyle of the femur from the inferior and superior surface viewpoints, respectively. FIG. 3C is a side view of the implant of FIG. 3 A. FIG. 3D is a view of the inferior surface of the implant; FIG. 3E is a view of the superior surface of the implant and FIG. 3F is a cross-section of the implant. FIG. 3G is an axial view of a femur with the implant installed thereon. FIG. 3H is an anterior view of the knee joint without the patella wherein the implant is installed on the femoral condyle. FIG. 31 is an anterior view of the knee joint with an implant of FIG. 3A implanted on the femoral condyle along with an implant suitable for the tibial plateau, such as that shown in FIG. 2. FIGS. 3J-K illustrate an alternate embodiment of a joint implant for use on a condyle of a femur further having at least one chamfer cut.

FIG. 4A illustrates an implant suitable for the femoral condyle according to the prior art. FIGS. 4B-I depict another implant suitable for placement on a femoral condyle. FIG. 4B is a slightly perspective view of the implant from the superior surface. FIG. 4C is a side view of the implant of FIG. 4B. FIG. 4D is a top view of the inferior surface of the implant; FIGS. 4E and F are perspective side views of the implant. FIG. 4G is an axial view of a femur with the implant installed thereon. FIG. 4H is an anterior view of the knee joint without the patella wherein the implant is installed on the femoral condyle. FIG. 4I is an anterior view of the knee joint with an implant of FIG. 4B implanted on the femoral condyle along with an implant suitable for the tibial plateau, such as that shown in FIG. 2.

FIGS. 5A-S are depictions of another implant suitable for placement on the femoral condyle. FIG. 5A is a top view of the inferior surface of the implant showing a chamfer cut. FIG. 5B is a slightly perspective view of the superior surface of the implant. FIG. 5C is a perspective side view of the implant from a first direction; FIG. 5D is a slightly perspective side view of the implant from a second direction. FIGS. 5E-F are side views of the implant showing the bearing loads; FIGS. 5G and H illustrate an alternative embodiment wherein the implant has lateral rails; FIG. 5I illustrates another embodiment wherein the implant has an anchoring keel. FIG. 5J is an axial view of a femur with the implant installed on the femoral condyles. FIG. 5K is an anterior view of the knee joint without the patella wherein the implant is installed on the femoral condyle.

FIG. 5L is an anterior view of the knee joint with an implant of FIG. 5A implanted on the femoral condyles along with an implant suitable for the tibial plateau, such as that shown in FIG. 2. FIGS. 5M-N depicts a device implanted within the knee joint. FIG. 50 depicts an alternate embodiment of the device which accommodates an partial removal of the condyle. FIGS. 5P-S illustrate alternative embodiments of the implant having one or more chamfer cuts.

FIGS. 6A-G illustrate a device as shown in FIG. 5 along with a graphical representation of the cross-sectional data points comprising the surface map.

FIGS. 7A-C illustrate an alternate design of a device, suitable for a portion of the femoral condyle, having a two piece configuration.

FIGS. 8A-J depict a whole patella (FIG. 8A) and a patella that has been cut in order to install an implant (FIG. 8B). A top and side view of a suitable patella implant is shown (FIGS. 8C-D), and an illustration of the implant superimposed on a whole patella is shown to illustrate the location of the implant dome relative to the patellar ridge. FIGS. 8E-F illustrate the implant superimposed over a patella. FIGS. 8G-J illustrate an alternate design for the patella implant based on a blank (FIG. 8G).

FIGS. 9A-C depict representative side views of a knee joint with any of the devices taught installed therein. FIG. 9A depicts the knee with a condyle implant and a patella implant. FIG. 9B depicts an alternate view of the knee with a condyle implant and a patella implant wherein the condyle implant covers a greater portion of the surface of the condyle in the posterior direction. FIG. 9C illustrates a knee joint wherein the implant is provided on the condyle, the patella and the tibial plateau.

FIGS. 10A-D depict a frontal view of the knee joint with any of the devices taught installed therein. FIG. 10A depicts the knee with a tibial implant. FIG. 10B depicts the knee with a condyle implant. FIG. 10C depicts a knee with a tibial implant and a condyle implant. FIG. 10C depicts a knee with a bicompartmental condyle implant and a tibial implant.

FIG. 11A shows a joint implant that includes a mobile bearing, in accordance with one embodiment of the invention. FIGS. 11B-J show exemplary external surfaces of the joint implant (e.g., facing the femur in a knee implant), in accordance with various embodiments of the invention.

FIGS. 12A-E show anchoring mechanisms for a joint implant that includes a mobile bearing, in accordance with various embodiments of the invention.

FIGS. 13A-G show bearing surfaces for a joint implant that includes a mobile bearing, in accordance with various embodiments of the invention.

FIGS. 14A-N shows perimeter, keel and peg configurations for the first component of an implant that includes a mobile bearing, in accordance with various embodiments of the invention.

FIGS. 15A-D show top surface radius configurations for a mobile bearing joint implant, in accordance with various embodiments of the invention.

FIGS. 16A-J show bearing and other surface configurations for a mobile bearing joint implant, in accordance with various embodiments of the invention.

FIGS. 17A-D show mobile bearing joint implant wherein the bearing component of the joint implant is slideably engaged with the first component, in accordance with various embodiments of the invention. FIGS. 17E-L show exemplary locations and configurations of the recessed slot of the first component, in accordance with various embodiments of the invention.

FIGS. 18A-F show mobile bearing joint devices having a stop restricting motion of the bearing component in one or more dimensions, in accordance with various embodiments of the invention.

FIGS. 19A-E show mobile bearing joint implant that include a stop, in accordance with various embodiments of the invention.

FIGS. 20A-C show varying shapes of bearing and first components of a mobile bearing joint implant, in accordance with various embodiments of the invention.

### DETAILED DESCRIPTION OF SPECIFIC EMBODIMENTS

The following description is presented to enable any person skilled in the art to make and use the invention. Various modifications to the embodiments described will be readily apparent to those skilled in the art, and the generic principles defined herein can be applied to other embodiments and applications without departing from the spirit and scope of the present invention as defined by the appended claims. Thus, the present invention is not intended to be limited to the embodiments shown, but is to be accorded the widest scope consistent with the principles and features disclosed herein. To the extent necessary to achieve a complete understanding of the invention disclosed, the specification and drawings of all issued patents, patent publications, and patent applications cited in this application are incorporated herein by reference.

As will be appreciated by those of skill in the art, methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

The practice of the present invention can employ, unless otherwise indicated, conventional and digital methods of x-ray imaging and processing, x-ray tomosynthesis, ultrasound including A-scan, B-scan and C-scan, computed tomography (CT scan), magnetic resonance imaging (MRI), optical coherence tomography, single photon emission tomography (SPECT) and positron emission tomography (PET) within the skill of the art. Such techniques are explained fully in the literature and need not be described herein. See, e.g., X-Ray Structure Determination: A Practical Guide, 2nd Edition, editors Stout and Jensen, 1989, John Wiley & Sons, publisher; Body CT: A Practical Approach, editor Slone, 1999, McGraw-Hill publisher; X-ray Diagnosis: A Physician's Approach, editor Lam, 1998 Springer-Verlag, publisher; and Dental Radiology: Understanding the X-Ray Image, editor Laetitia Brocklebank 1997, Oxford University Press publisher. See also, The Essential Physics of Medical Imaging (2^{nd} Ed.), Jerrold T. Bushberg, et al.

The present invention provides methods and compositions for repairing joints, particularly for repairing articular cartilage and for facilitating the integration of a wide variety of cartilage repair materials into a subject. Among other things, the techniques described herein allow for the customization of cartilage repair material to suit a particular subject, for example in terms of size, cartilage thickness and/or curvature. When the shape (e.g., size, thickness and/or curvature) of the articular cartilage surface is an exact or near anatomic fit with the non-damaged cartilage or with the subject's original cartilage, the success of repair is enhanced. The repair material can be shaped prior to implantation and such shaping can be based, for example, on electronic images that provide information regarding curvature or thickness of any “normal” cartilage surrounding the defect and/or on curvature of the bone underlying the defect. Thus, the current invention provides, among other things, for minimally invasive methods for partial joint replacement. The methods will require only minimal or, in some instances, no loss in bone stock. Additionally, unlike with current techniques, the methods described herein will help to restore the integrity of the articular surface by achieving an exact or near anatomic match between the implant and the surrounding or adjacent cartilage and/or subchondral bone.

Advantages of the present invention can include, but are not limited to, (i) customization of joint repair, thereby enhancing the efficacy and comfort level for the patient following the repair procedure; (ii) eliminating the need for a surgeon to measure the defect to be repaired intraoperatively in some embodiments; (iii) eliminating the need for a surgeon to shape the material during the implantation procedure; (iv) providing methods of evaluating curvature of the repair material based on bone or tissue images or based on intraoperative probing techniques; (v) providing methods of repairing joints with only minimal or, in some instances, no loss in bone stock; (vi) improving postoperative joint congruity; (vii) improving the postoperative patient recovery in some embodiments and (viii) improving postoperative function, such as range of motion.

Thus, the methods described herein allow for the design and use of joint repair material that more precisely fits the defect (e.g., site of implantation) or the articular surface(s) and, accordingly, provides improved repair of the joint,

### I. Assessment of Joints and Alignment

The methods and compositions described herein can be used to treat defects resulting from disease of the cartilage (e.g., osteoarthritis), bone damage, cartilage damage, trauma, and/or degeneration due to overuse or age. The invention allows, among other things, a health practitioner to evaluate and treat such defects. The size, volume and shape of the area of interest can include only the region of cartilage that has the defect, but preferably will also include contiguous parts of the cartilage surrounding the cartilage defect.

As will be appreciated by those of skill in the art, size, curvature and/or thickness measurements can be obtained using any suitable technique. For example, one-dimensional, two-dimensional, and/or three-dimensional measurements can be obtained using suitable mechanical means, laser devices, electromagnetic or optical tracking systems, molds, materials applied to the articular surface that harden and “memorize the surface contour,” and/or one or more imaging techniques known in the art. Measurements can be obtained non-invasively and/or intraoperatively (e.g., using a probe or other surgical device). As will be appreciated by those of skill in the art, the thickness of the repair device can vary at any given point depending upon patient's anatomy and/or the depth of the damage to the cartilage and/or bone to be corrected at any particular location on an articular surface.

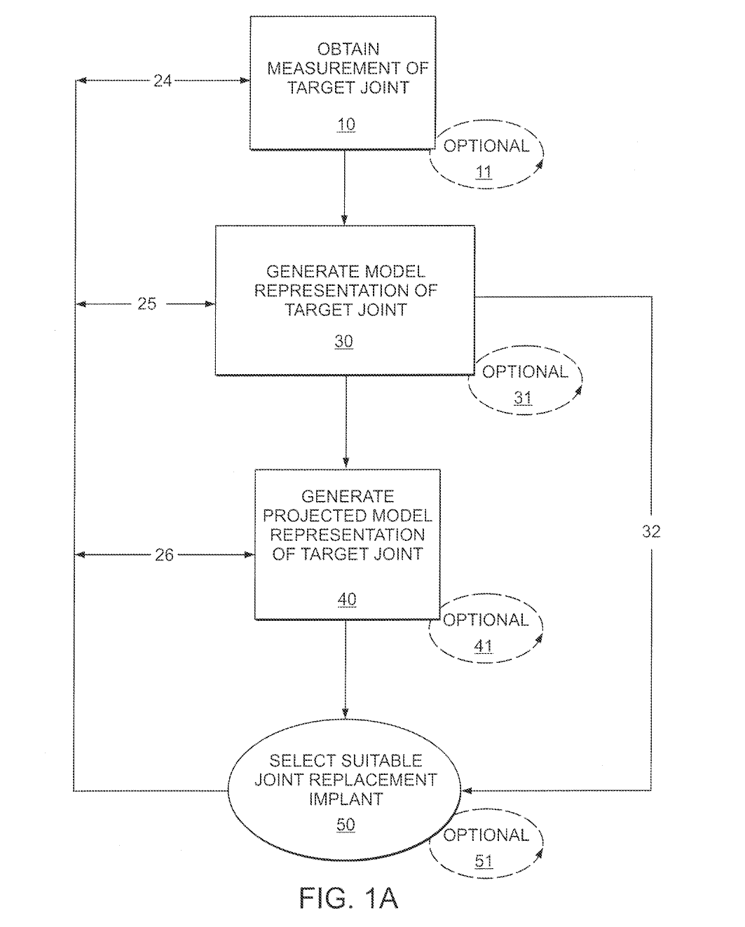

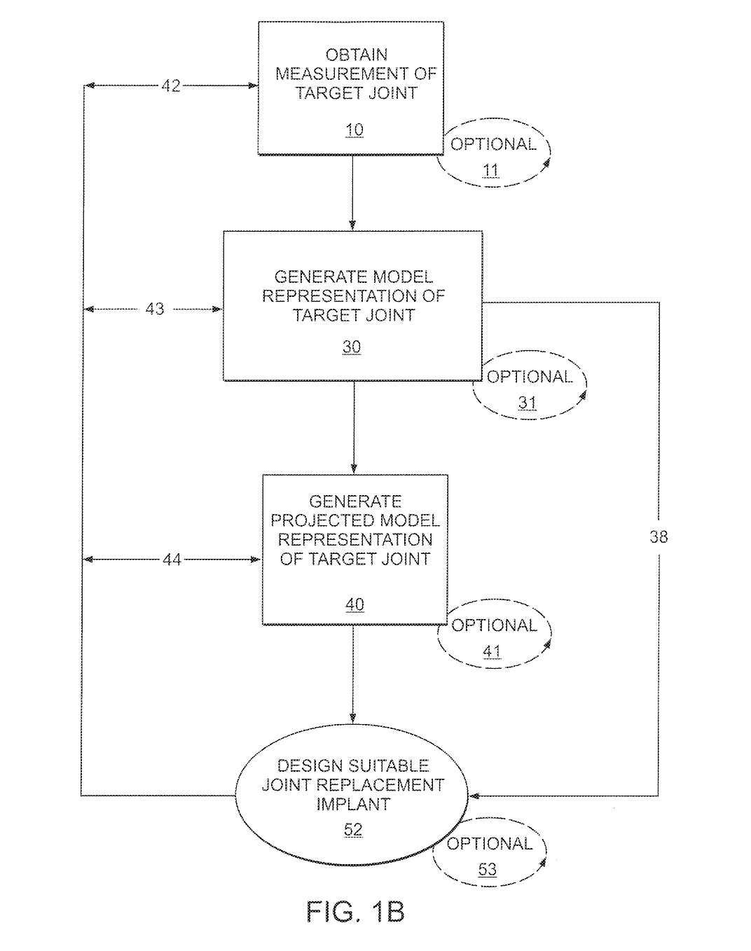

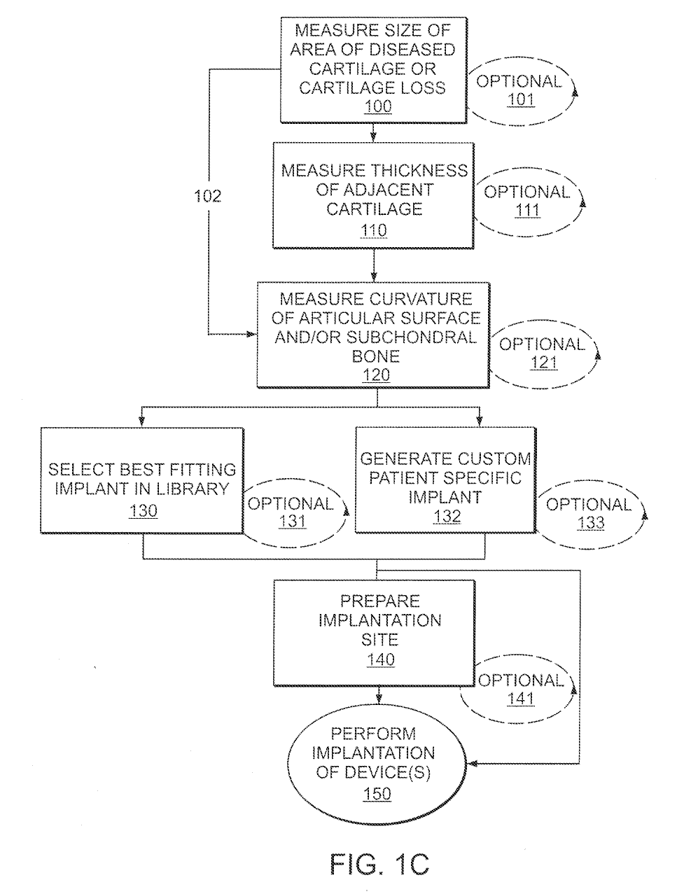

FIG. 1A is a flow chart showing steps taken by a practitioner in assessing a joint. First, a practitioner obtains a measurement of a target joint 10. The step of obtaining a measurement can be accomplished by taking an image of the joint. This step can be repeated, as necessary, 11 to obtain a plurality of images in order to further refine the joint assessment process. Once the practitioner has obtained the necessary measurements, the information is used to generate a model representation of the target joint being assessed 30. This model representation can be in the form of a topographical map or image. The model representation of the joint can be in one, two, or three dimensions. It can include a physical model. More than one model can be created 31, if desired. Either the original model, or a subsequently created model, or both can be used. After the model representation of the joint is generated 30, the practitioner can optionally generate a projected model representation of the target joint in a corrected condition 40, e.g., from the existing cartilage on the joint surface, by providing a mirror of the opposing joint surface, or a combination thereof. Again, this step can be repeated 41, as necessary or desired. Using the difference between the topographical condition of the joint and the projected image of the joint, the practitioner can then select a joint implant 50 that is suitable to achieve the corrected joint anatomy. As will be appreciated by those of skill in the art, the selection process 50 can be repeated 51 as often as desired to achieve the desired result. Additionally, it is contemplated that a practitioner can obtain a measurement of a target joint 10 by obtaining, for example, an x-ray, and then select a suitable joint replacement implant 50.

As will be appreciated by those of skill in the art, the practitioner can proceed directly from the step of generating a model representation of the target joint 30 to the step of selecting a suitable joint replacement implant 50 as shown by the arrow 32. Additionally, following selection of suitable joint replacement implant 50, the steps of obtaining measurement of target joint 10, generating model representation of target joint 30 and generating projected model 40, can be repeated in series or parallel as shown by the flow 24, 25, 26.

FIG. 1B is an alternate flow chart showing steps taken by a practitioner in assessing a joint. First, a practitioner obtains a measurement of a target joint 10. The step of obtaining a measurement can be accomplished by taking an image of the joint. This step can be repeated, as necessary, 11 to obtain a plurality of images in order to further refine the joint assessment process. Once the practitioner has obtained the necessary measurements, the information is used to generate a model representation of the target joint being assessed 30. This model representation can be in the form of a topographical map or image. The model representation of the joint can be in one, two, or three dimensions. The process can be repeated 31 as necessary or desired. It can include a physical model. After the model representation of the joint is assessed 30, the practitioner can optionally generate a projected model representation of the target joint in a corrected condition 40. This step can be repeated 41 as necessary or desired. Using the difference between the topographical condition of the joint and the projected image of the joint, the practitioner can then design a joint implant 52 that is suitable to achieve the corrected joint anatomy, repeating the design process 53 as often as necessary to achieve the desired implant design. The practitioner can also assess whether providing additional features, such as rails, keels, lips, pegs, cruciate stems, or anchors, cross-bars, etc. will enhance the implants' performance in the target joint.

As will be appreciated by those of skill in the art, the practitioner can proceed directly from the step of generating a model representation of the target joint 30 to the step of designing a suitable joint replacement implant 52 as shown by the arrow 38. Similar to the flow shown above, following the design of a suitable joint replacement implant 52, the steps of obtaining measurement of target joint 10, generating model representation of target joint 30 and generating projected model 40, can be repeated in series or parallel as shown by the flow 42, 43, 44.

FIG. 1C is a flow chart illustrating the process of selecting an implant for a patient. First, using the techniques described above or those suitable and known in the art at the time the invention is practiced, the size of area of diseased cartilage or cartilage loss is measured 100. This step can be repeated multiple times 101, as desired. Once the size of the cartilage defect is measured, the thickness of adjacent cartilage can optionally be measured 110. This process can also be repeated as desired 111. Either after measuring the cartilage loss or measuring the thickness of adjacent cartilage, the curvature of the articular surface is then measured 120. Alternatively, the subchondral bone can be measured. As will be appreciated measurements can be taken of the surface of the joint being repaired, or of the mating surface in order to facilitate development of the best design for the implant surface.

Once the surfaces have been measured, the user either selects the best fitting implant contained in a library of implants 130 or generates a patient-specific implant 132. These steps can be repeated as desired or necessary to achieve the best fitting implant for a patient, 131, 133. As will be appreciated by those of skill in the art, the process of selecting or designing an implant can be tested against the information contained in the MRI or x-ray of the patient to ensure that the surfaces of the device achieves a good fit relative to the patient's joint surface. Testing can be accomplished by, for example, superimposing the implant image over the image for the patient's joint. Once it has been determined that a suitable implant has been selected or designed, the implant site can be prepared 140, for example by removing cartilage or bone from the joint surface, or the implant can be placed into the joint 150.

The joint implant selected or designed achieves anatomic or near anatomic fit with the existing surface of the joint while presenting a mating surface for the opposing joint surface that replicates the natural joint anatomy. In this instance, both the existing surface of the joint can be assessed as well as the desired resulting surface of the joint. This technique is particularly useful for implants that are not anchored into the bone.

As will be appreciated by those of skill in the art, the physician, or other person practicing the invention, can obtain a measurement of a target joint 10 and then either design 52 or select 50 a suitable joint replacement implant.

### II. Repair Materials

A wide variety of materials find use in the practice of the present invention, including, but not limited to, plastics, metals, crystal free metals, ceramics, biological materials (e.g., collagen or other extracellular matrix materials), hydroxyapatite, cells (e.g., stem cells, chondrocyte cells or the like), or combinations thereof. Based on the information (e.g., measurements) obtained regarding the defect and the articular surface and/or the subchondral bone, a repair material can be formed or selected. Further, using one or more of these techniques described herein, a cartilage replacement or regenerating material having a curvature that will fit into a particular cartilage defect, will follow the contour and shape of the articular surface, and will match the thickness of the surrounding cartilage. The repair material can include any combination of materials, and typically includes at least one non-pliable material, for example materials that are not easily bent or changed.

### A. Metal and Polymeric Repair Materials

Currently, joint repair systems often employ metal and/or polymeric materials including, for example, prostheses which are anchored into the underlying bone (e.g., a femur in the case of a knee prosthesis). See, e.g., U.S. Pat. No. 6,203,576 to Afriat, et al. issued Mar. 20, 2001 and U.S. Pat. No. 6,322,588 to Ogle, et al. issued Nov. 27, 2001, and references cited therein. A wide-variety of metals are useful in the practice of the present invention, and can be selected based on any criteria. For example, material selection can be based on resiliency to impart a desired degree of rigidity. Non-limiting examples of suitable metals include silver, gold, platinum, palladium, iridium, copper, tin, lead, antimony, bismuth, zinc, titanium, cobalt, stainless steel, nickel, iron alloys, cobalt alloys, such as Elgiloy®, a cobalt-chromium-nickel alloy, and MP35N, a nickel-cobalt-chromium-molybdenum alloy, and Nitinol™, a nickel-titanium alloy, aluminum, manganese, iron, tantalum, crystal free metals, such as Liquidmetal® alloys (available from LiquidMetal Technologies, www.liquidmetal.com), other metals that can slowly form polyvalent metal ions, for example to inhibit calcification of implanted substrates in contact with a patient's bodily fluids or tissues, and combinations thereof.

Suitable synthetic polymers include, without limitation, polyamides (e.g., nylon), polyesters, polystyrenes, polyacrylates, vinyl polymers (e.g., polyethylene, polytetrafluoroethylene, polypropylene and polyvinyl chloride), polycarbonates, polyurethanes, poly dimethyl siloxanes, cellulose acetates, polymethyl methacrylates, polyether ether ketones, ethylene vinyl acetates, polysulfones, nitrocelluloses, similar copolymers and mixtures thereof. Bioresorbable synthetic polymers can also be used such as dextran, hydroxyethyl starch, derivatives of gelatin, polyvinylpyrrolidone, polyvinyl alcohol, poly[N-(2-hydroxypropyl)methacrylamide], poly(hydroxy acids), poly(epsilon-caprolactone), polylactic acid, polyglycolic acid, poly(dimethyl glycolic acid), poly(hydroxy butyrate), and similar copolymers can also be used.

Other materials would also be appropriate, for example, the polyketone known as polyetheretherketone (PEEK™). This includes the material PEEK 450G, which is an unfilled PEEK approved for medical implantation available from Victrex of Lancashire, Great Britain. (Victrex is located at www.matweb.com or see Boedeker www.boedeker.com). Other sources of this material include Gharda located in Panoli, India (www.ghardapolymers.com).

It should be noted that the material selected can also be filled. For example, other grades of PEEK are also available and contemplated, such as 30% glass-filled or 30% carbon filled, provided such materials are cleared for use in implantable devices by the FDA, or other regulatory body. Glass filled PEEK reduces the expansion rate and increases the flexural modulus of PEEK relative to that portion which is unfilled. The resulting product is known to be ideal for improved strength, stiffness, or stability. Carbon filled PEEK is known to enhance the compressive strength and stiffness of PEEK and lower its expansion rate. Carbon filled PEEK offers wear resistance and load carrying capability.

As will be appreciated, other suitable similarly biocompatible thermoplastic or thermoplastic polycondensate materials that resist fatigue, have good memory, are flexible, and/or deflectable have very low moisture absorption, and good wear and/or abrasion resistance, can be used without departing from the scope of the invention. The implant can also be comprised of polyetherketoneketone (PEKK).

Other materials that can be used include polyetherketone (PEK), polyetherketoneetherketoneketone (PEKEKK), and polyetheretherketoneketone (PEEKK), and generally a polyaryletheretherketone. Further other polyketones can be used as well as other thermoplastics.

Reference to appropriate polymers that can be used for the implant can be made to the following documents, all of which are incorporated herein by reference. These documents include: PCT Publication WO 02/02158 A1, dated Jan. 10, 2002 and entitled Bio-Compatible Polymeric Materials; PCT Publication WO 02/00275 A1, dated Jan. 3, 2002 and entitled Bio-Compatible Polymeric Materials; and PCT Publication WO 02/00270 A1, dated Jan. 3, 2002 and entitled Bio-Compatible Polymeric Materials.

The polymers can be prepared by any of a variety of approaches including conventional polymer processing methods. Preferred approaches include, for example, injection molding, which is suitable for the production of polymer components with significant structural features, and rapid prototyping approaches, such as reaction injection molding and stereo-lithography. The substrate can be textured or made porous by either physical abrasion or chemical alteration to facilitate incorporation of the metal coating. Other processes are also appropriate, such as extrusion, injection, compression molding and/or machining techniques. Typically, the polymer is chosen for its physical and mechanical properties and is suitable for carrying and spreading the physical load between the joint surfaces.

More than one metal and/or polymer can be used in combination with each other. For example, one or more metal-containing substrates can be coated with polymers in one or more regions or, alternatively, one or more polymer-containing substrate can be coated in one or more regions with one or more metals.

The system or prosthesis can be porous or porous coated. The porous surface components can be made of various materials including metals, ceramics, and polymers. These surface components can, in turn, be secured by various means to a multitude of structural cores formed of various metals. Suitable porous coatings include, but are not limited to, metal, ceramic, polymeric (e.g., biologically neutral elastomers such as silicone rubber, polyethylene terephthalate and/or combinations thereof) or combinations thereof. See, e.g., U.S. Pat. No. 3,605,123 to Hahn, issued Sep. 20, 1971. U.S. Pat. No. 3,808,606 to Tronzo issued May 7, 1974 and U.S. Pat. No. 3,843,975 to Tronzo issued Oct. 29, 1974; U.S. Pat. No. 3,314,420 to Smith issued Apr. 18, 1967; U.S. Pat. No. 3,987,499 to Scharbach issued Oct. 26, 1976; and German Offenlegungsschrift 2,306,552. There can be more than one coating layer and the layers can have the same or different porosities. See, e.g., U.S. Pat. No. 3,938,198 to Kahn, et al., issued Feb. 17, 1976.

The coating can be applied by surrounding a core with powdered polymer and heating until cured to form a coating with an internal network of interconnected pores. The tortuosity of the pores (e.g., a measure of length to diameter of the paths through the pores) can be important in evaluating the probable success of such a coating in use on a prosthetic device. See, also, U.S. Pat. No. 4,213,816 to Morris issued Jul. 22, 1980. The porous coating can be applied in the form of a powder and the article as a whole subjected to an elevated temperature that bonds the powder to the substrate. Selection of suitable polymers and/or powder coatings can be determined in view of the teachings and references cited herein, for example based on the melt index of each.

### B. Biological Repair Material

Repair materials can also include one or more biological material either alone or in combination with non-biological materials. For example, any base material can be designed or shaped and suitable cartilage replacement or regenerating material(s) such as fetal cartilage cells can be applied to be the base. The cells can be then be grown in conjunction with the base until the thickness (and/or curvature) of the cartilage surrounding the cartilage defect has been reached. Conditions for growing cells (e.g., chondrocytes) on various substrates in culture, ex vivo and in vivo are described, for example, in U.S. Pat. Nos. 5,478,739 to Slivka et al. issued Dec. 26, 1995; 5,842,477 to Naughton et al. issued Dec. 1, 1998; 6,283,980 to Vibe-Hansen et al., issued Sep. 4, 2001, and 6,365,405 to Salzmann et al. issued Apr. 2, 2002. Non-limiting examples of suitable substrates include plastic, tissue scaffold, a bone replacement material (e.g., a hydroxyapatite, a bioresorbable material), or any other material suitable for growing a cartilage replacement or regenerating material on it.

Biological polymers can be naturally occurring or produced in vitro by fermentation and the like. Suitable biological polymers include, without limitation, collagen, elastin, silk, keratin, gelatin, polyamino acids, cat gut sutures, polysaccharides (e.g., cellulose and starch) and mixtures thereof. Biological polymers can be bioresorbable.

Biological materials used in the methods described herein can be autografts (from the same subject); allografts (from another individual of the same species) and/or xenografts (from another species). See, also, International Patent Publications WO 02/22014 to Alexander et al. published Mar. 21, 2002 and WO 97/27885 to Lee published Aug. 7, 1997. In certain embodiments autologous materials are preferred, as they can carry a reduced risk of immunological complications to the host, including re-absorption of the materials, inflammation and/or scarring of the tissues surrounding the implant site.

In one embodiment of the invention, a probe is used to harvest tissue from a donor site and to prepare a recipient site. The donor site can be located in a xenograft, an allograft or an autograft. The probe is used to achieve a good anatomic match between the donor tissue sample and the recipient site. The probe is specifically designed to achieve a seamless or near seamless match between the donor tissue sample and the recipient site. The probe can, for example, be cylindrical. The distal end of the probe is typically sharp in order to facilitate tissue penetration. Additionally, the distal end of the probe is typically hollow in order to accept the tissue. The probe can have an edge at a defined distance from its distal end, e.g. at 1 cm distance from the distal end and the edge can be used to achieve a defined depth of tissue penetration for harvesting. The edge can be external or can be inside the hollow portion of the probe. For example, an orthopedic surgeon can take the probe and advance it with physical pressure into the cartilage, the subchondral bone and the underlying marrow in the case of a joint such as a knee joint. The surgeon can advance the probe until the external or internal edge reaches the cartilage surface. At that point, the edge will prevent further tissue penetration thereby achieving a constant and reproducible tissue penetration. The distal end of the probe can include one or more blades, saw-like structures, or tissue cutting mechanism. For example, the distal end of the probe can include an iris-like mechanism consisting of several small blades. The blade or blades can be moved using a manual, motorized or electrical mechanism thereby cutting through the tissue and separating the tissue sample from the underlying tissue. Typically, this will be repeated in the donor and the recipient. In the case of an iris-shaped blade mechanism, the individual blades can be moved so as to close the iris thereby separating the tissue sample from the donor site.

In another embodiment of the invention, a laser device or a radio frequency device can be integrated inside the distal end of the probe. The laser device or the radio frequency device can be used to cut through the tissue and to separate the tissue sample from the underlying tissue.

In one embodiment of the invention, the same probe can be used in the donor and in the recipient. In another embodiment, similarly shaped probes of slightly different physical dimensions can be used. For example, the probe used in the recipient can be slightly smaller than that used in the donor thereby achieving a tight fit between the tissue sample or tissue transplant and the recipient site. The probe used in the recipient can also be slightly shorter than that used in the donor thereby correcting for any tissue lost during the separation or cutting of the tissue sample from the underlying tissue in the donor material.

Any biological repair material can be sterilized to inactivate biological contaminants such as bacteria, viruses, yeasts, molds, mycoplasmas and parasites. Sterilization can be performed using any suitable technique, for example radiation, such as gamma radiation.

Any of the biological materials described herein can be harvested with use of a robotic device. The robotic device can use information from an electronic image for tissue harvesting.

In certain embodiments, the cartilage replacement material has a particular biochemical composition. For instance, the biochemical composition of the cartilage surrounding a defect can be assessed by taking tissue samples and chemical analysis or by imaging techniques. For example, WO 02/22014 to Alexander describes the use of gadolinium for imaging of articular cartilage to monitor glycosaminoglycan content within the cartilage. The cartilage replacement or regenerating material can then be made or cultured in a manner, to achieve a biochemical composition similar to that of the cartilage surrounding the implantation site. The culture conditions used to achieve the desired biochemical compositions can include, for example, varying concentrations. Biochemical composition of the cartilage replacement or regenerating material can, for example, be influenced by controlling concentrations and exposure times of certain nutrients and growth factors.

### III. Device Design

Using information on thickness and curvature of the cartilage and/or subchondral bone, a physical model of the surfaces of the articular cartilage and/or of the underlying bone can be created. This physical model can be representative of a limited area within the joint or it can encompass the entire joint. This model can also take into consideration the presence or absence of a meniscus as well as the presence or absence of some or all of the cartilage. For example, in the knee joint, the physical model can encompass only the medial or lateral femoral condyle, both femoral condyles and the notch region, the medial tibial plateau, the lateral tibia! plateau, the entire tibia! plateau, the medial patella, the lateral patella, the entire patella or the entire joint. The location of a diseased area of cartilage can be determined, for example using a 3D coordinate system or a 3D Euclidian distance as described in WO 02/22014.

In this way, the size of the defect to be repaired can be determined. This process takes into account that, for example, roughly 80% of patients have a healthy lateral component. As will be apparent, some, but not all, defects will include less than the entire cartilage. Thus, in one embodiment of the invention, the thickness of the normal or only mildly diseased cartilage surrounding one or more cartilage defects is measured. This thickness measurement can be obtained at a single point or, preferably, at multiple points, for example 2 point, 4-6 points, 7-10 points, more than 10 points or over the length of the entire remaining cartilage. Furthermore, once the size of the defect is determined, an appropriate therapy (e.g., articular repair system) can be selected such that as much as possible of the healthy, surrounding tissue is preserved.

In other embodiments, the curvature of the articular surface can be measured to design and/or shape the repair material. Further, both the thickness of the remaining cartilage and the curvature of the articular surface can be measured to design and/or shape the repair material. Alternatively, the curvature of the subchondral bone can be measured and the resultant measurement(s) can be used to either select or shape a cartilage replacement material. For example, the contour of the subchondral bone can be used to re-create a virtual cartilage surface: the margins of an area of diseased cartilage can be identified. The subchondral bone shape in the diseased areas can be measured. A virtual contour can then be created by copying the subchondral bone surface into the cartilage surface, whereby the copy of the subchondral bone surface connects the margins of the area of diseased cartilage. In shaping the device, the contours can be configured to mate with existing cartilage or to account for the removal of some or all of the cartilage.

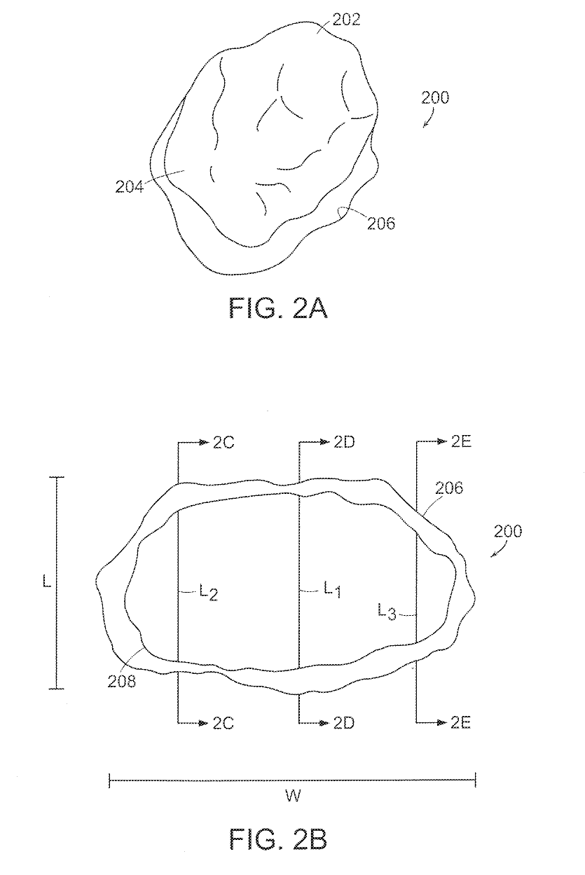

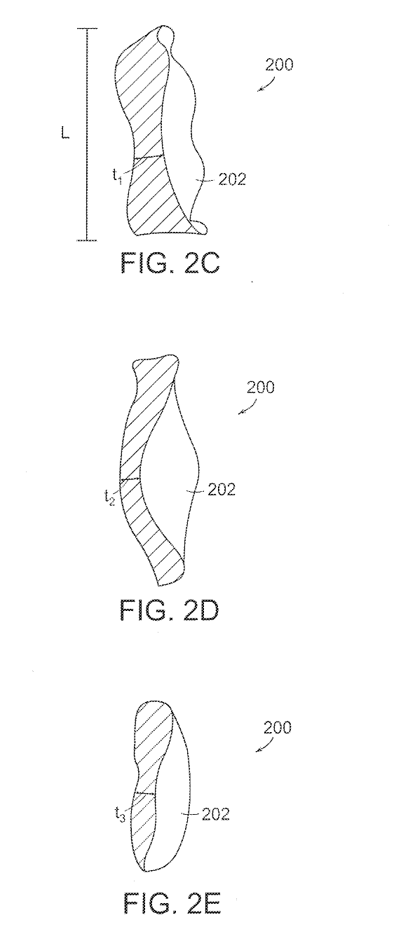

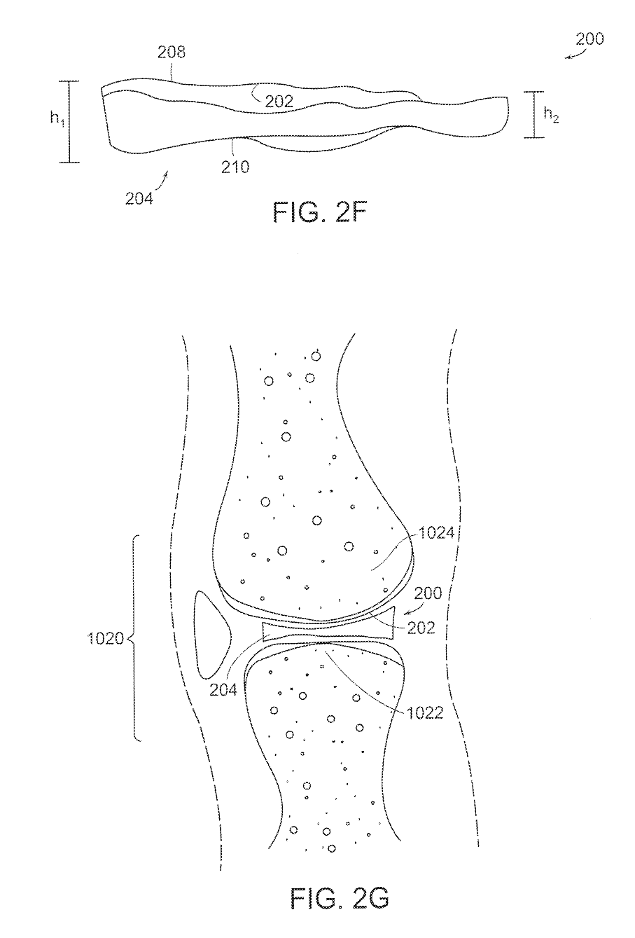

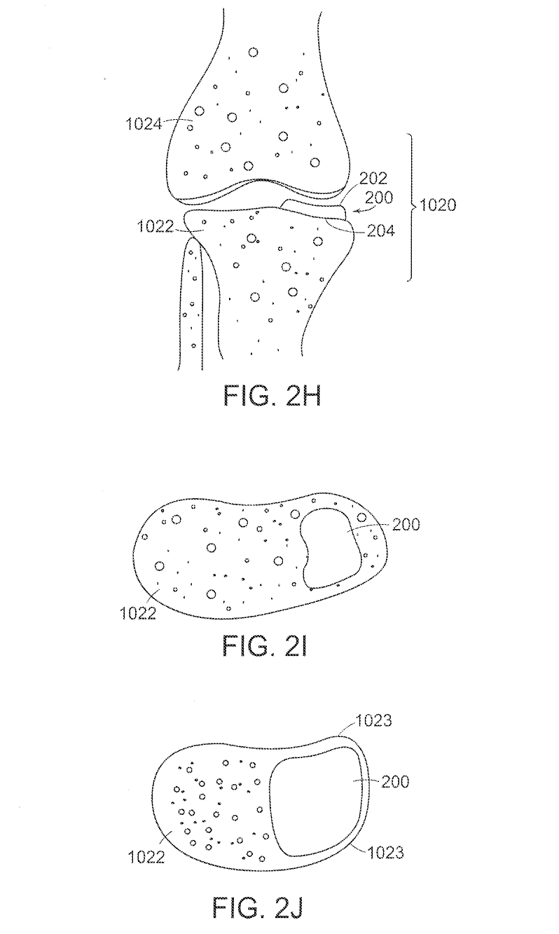

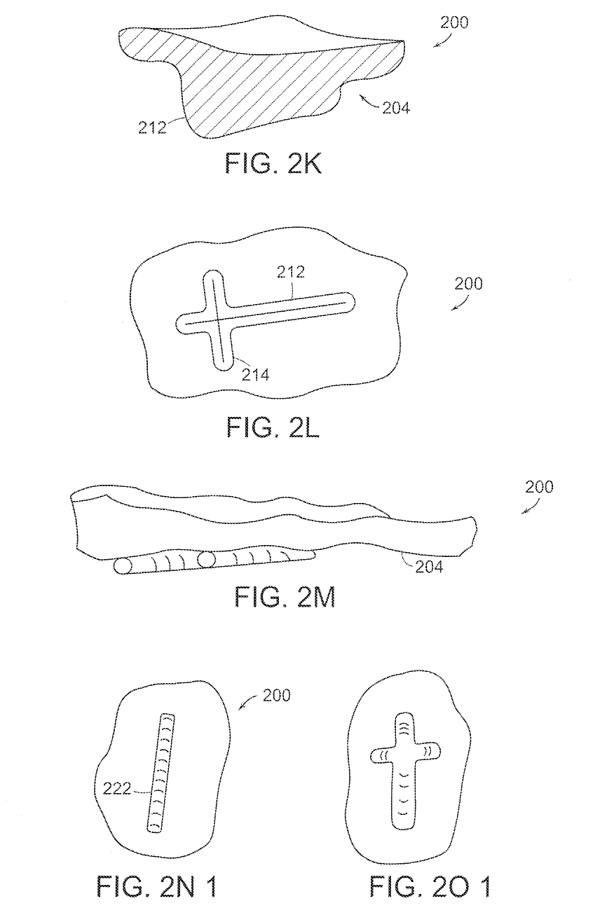

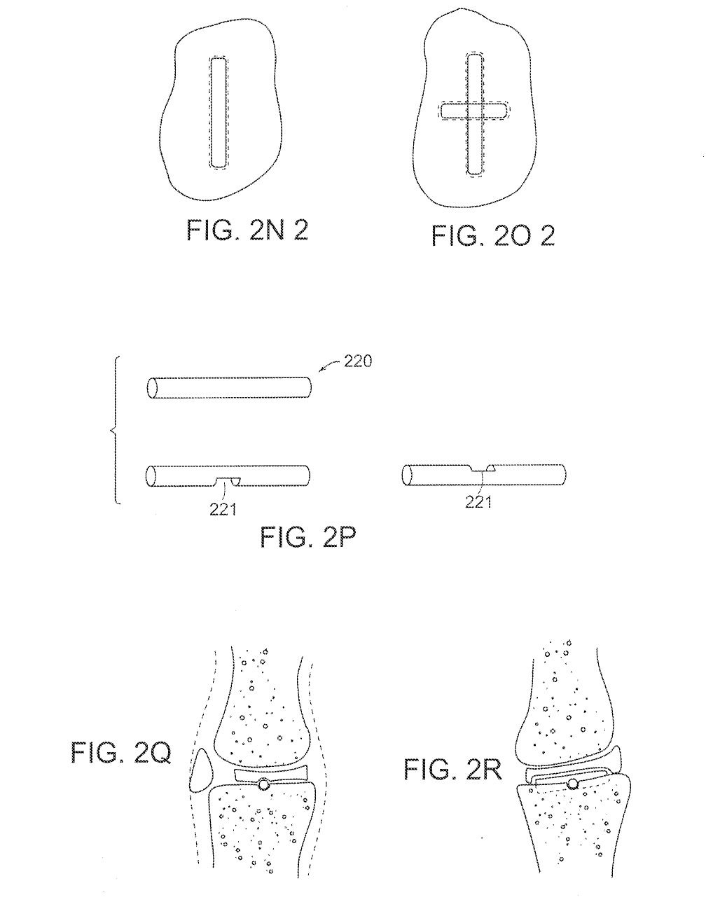

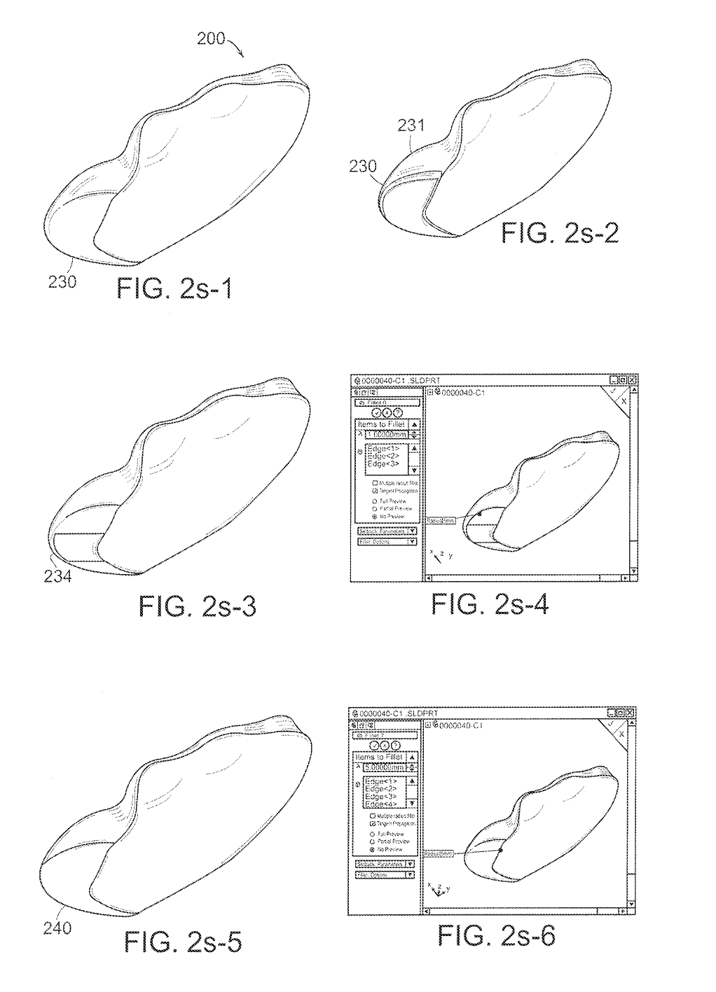

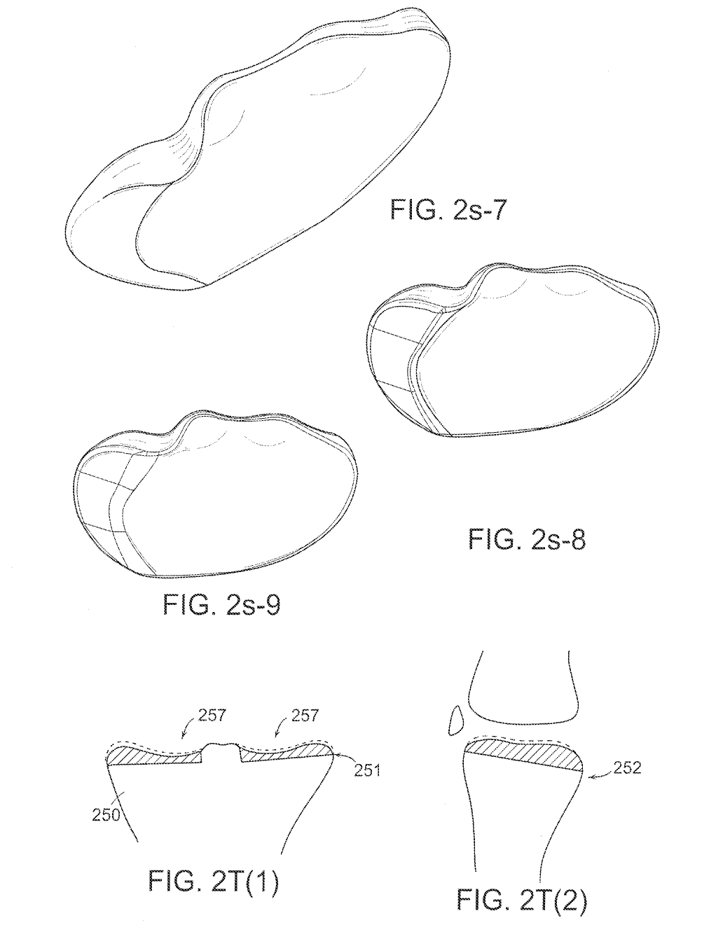

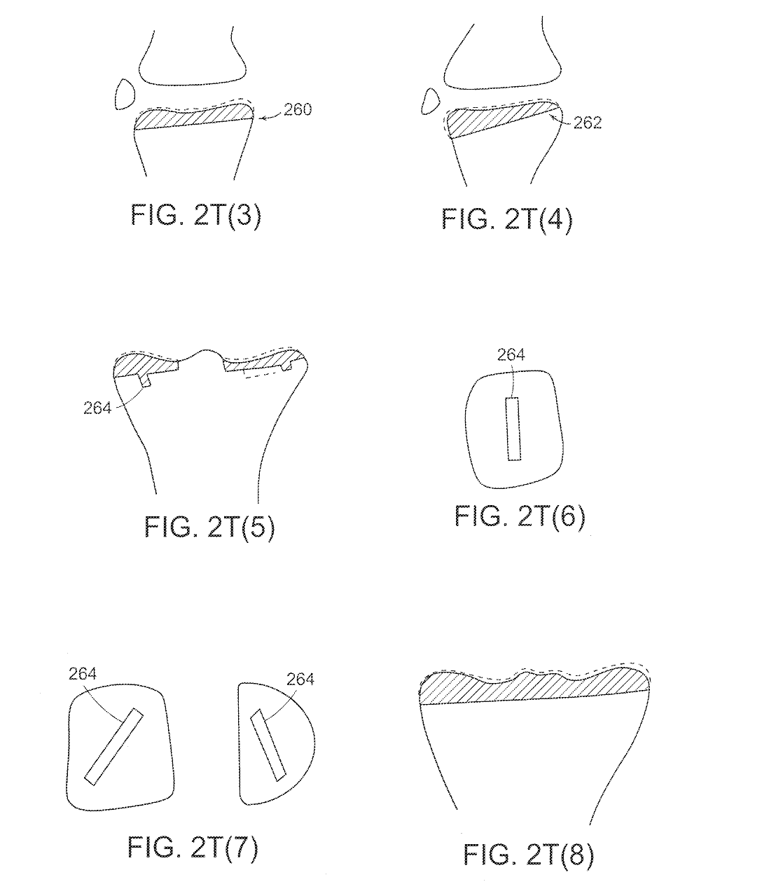

FIG. 2A shows a slightly perspective top view of a joint implant 200 of the invention suitable for implantation at the tibial plateau of the knee joint. As shown in FIG. 2A, the implant can be generated using, for example, a dual surface assessment, as described above with respect to FIGS. 1A and B.

The implant 200 has an upper surface 202, a lower surface 204 and a peripheral edge 206. The upper surface 202 is formed so that it forms a mating surface for receiving the opposing joint surface; in this instance partially concave to receive the femur. The concave surface can be variably concave such that it presents a surface to the opposing joint surface, e.g. a negative surface of the mating surface of the femur it communicates with. As will be appreciated by those of skill in the art, the negative impression, need not be a perfect one.

The upper surface 202 of the implant 200 can be shaped by any of a variety of means. For example, the upper surface 202 can be shaped by projecting the surface from the existing cartilage and/or bone surfaces on the tibial plateau, or it can be shaped to mirror the femoral condyle in order to optimize the complimentary surface of the implant when it engages the femoral condyle. Alternatively, the superior surface 202 can be configured to mate with an inferior surface of an implant configured for the opposing femoral condyle.

The lower surface 204 has a convex surface that matches, or nearly matches, the tibial plateau of the joint such that it creates an anatomic or near anatomic fit with the tibial plateau. Depending on the shape of the tibial plateau, the lower surface can be partially convex as well. Thus, the lower surface 204 presents a surface to the tibial plateau that fits within the existing surface. It can be formed to match the existing surface or to match the surface after articular resurfacing.

As will be appreciated by those of skill in the art, the convex surface of the lower surface 204 need not be perfectly convex. Rather, the lower surface 204 is more likely consist of convex and concave portions that fit within the existing surface of the tibial plateau or the re-surfaced plateau. Thus, the surface is essentially variably convex and concave.

FIG. 2B shows atop view of the joint implant of FIG. 2A. As shown in FIG. 2B the exterior shape 208 of the implant can be elongated. The elongated form can take a variety of shapes including elliptical, quasi-elliptical, race-track, etc. However, as will be appreciated the exterior dimension is typically irregular thus not forming a true geometric shape, e.g. ellipse. As will be appreciated by those of skill in the art, the actual exterior shape of an implant can vary depending on the nature of the joint defect to be corrected. Thus the ratio of the length L to the width W can vary from, for example, between 0.25 to 2.0, and more specifically from 0.5 to 1.5. As further shown in FIG. 2B, the length across an axis of the implant 200 varies when taken at points along the width of the implant. For example, as shown in FIG. 2B, L₁≠L₂≠L₃.

Turning now to FIGS. 2C-E, cross-sections of the implant shown in FIG. 2B are depicted along the lines of C-C, D-D, and E-E. The implant has a thickness t1, t2 and t3 respectively. As illustrated by the cross-sections, the thickness of the implant varies along both its length L and width W. The actual thickness at a particular location of the implant 200 is a function of the thickness of the cartilage and/or bone to be replaced and the joint mating surface to be replicated. Further, the profile of the implant 200 at any location along its length L or width W is a function of the cartilage and/or bone to be replaced.

FIG. 2F is a lateral view of the implant 200 of FIG. 2A. In this instance, the height of the implant 200 at a first end h₁ is different than the height of the implant at a second end h₂. Further the upper edge 208 can have an overall slope in a downward direction. However, as illustrated the actual slope of the upper edge 208 varies along its length and can, in some instances, be a positive slope. Further the lower edge 210 can have an overall slope in a downward direction. As illustrated the actual slope of the lower edge 210 varies along its length and can, in some instances, be a positive slope. As will be appreciated by those of skill in the art, depending on the anatomy of an individual patient, an implant can be created wherein h₁ and h₂ are equivalent, or substantially equivalent without departing from the scope of the invention.

FIG. 2G is a cross-section taken along a sagittal plane in a body showing the implant 200 implanted within a knee joint 1020 such that the lower surface 204 of the implant 200 lies on the tibial plateau 1022 and the femur 1024 rests on the upper surface 202 of the implant 200. FIG. 2H is a cross-section taken along a coronal plane in a body showing the implant 200 implanted within a knee joint 1020. As is apparent from this view, the implant 200 is positioned so that it fits within a superior articular surface 224. As will be appreciated by those of skill in the art, the articular surface could be the medial or lateral facet, as needed.

FIG. 2I is a view along an axial plane of the body showing the implant 200 implanted within a knee joint 1020 showing the view taken from an aerial, or upper, view. FIG. 2J is a view of an alternate embodiment where the implant is a bit larger such that it extends closer to the bone medially, i.e. towards the edge 1023 of the tibial plateau, as well as extending anteriorly and posteriorly.

FIG. 2K is a cross-section of an implant 200 of the invention according to an alternate embodiment. In this embodiment, the lower surface 204 further includes a joint anchor 212. As illustrated in this embodiment, the joint anchor 212 forms a protrusion, keel or vertical member that extends from the lower surface 204 of the implant 200 and projects into, for example, the bone of the joint. As will be appreciated by those of skill in the art, the keel can be perpendicular or lie within a plane of the body.

Additionally, as shown in FIG. 2L the joint anchor 212 can have a cross-member 214 so that from a bottom perspective, the joint anchor 212 has the appearance of a cross or an “x.” As will be appreciated by those of skill in the art, the joint anchor 212 could take on a variety of other forms while still accomplishing the same objective of providing increased stability of the implant 200 in the joint. These forms include, but are not limited to, pins, bulbs, balls, teeth, etc. Additionally, one or more joint anchors 212 can be provided as desired. FIGS. 2M and N illustrate cross-sections of alternate embodiments of a dual component implant from a side view and a front view.

In an alternate embodiment shown in FIG. 2M it may be desirable to provide a one or more cross-members 220 on the lower surface 204 in order to provide a bit of translation movement of the implant relative to the surface of the femur, or femur implant. In that event, the cross-member can be formed integral to the surface of the implant or can be one or more separate pieces that fit within a groove 222 on the lower surface 204 of the implant 200. The groove can form a single channel as shown in FIG. 2N1, or can have more than one channel as shown in FIG. 2N2. In either event, the cross-bar then fits within the channel as shown in FIGS. 2N1-N2. The cross-bar members 220 can form a solid or hollow tube or pipe structure as shown in FIG. 2P. Where two, or more, tubes 220 communicate to provide translation, a groove 221 can be provided along the surface of one or both cross-members to interlock the tubes into a cross-bar member further stabilizing the motion of the cross-bar relative to the implant 200. As will be appreciated by those of skill in the art, the cross-bar member 220 can be formed integrally with the implant without departing from the scope of the invention.

As shown in FIGS. 2Q-R, it is anticipated that the surface of the tibial plateau will be prepared by forming channels thereon to receive the cross-bar members. Thus facilitating the ability of the implant to seat securely within the joint while still providing movement about an axis when the knee joint is in motion.

FIG. 2S(1-9) illustrate an alternate embodiment of implant 200. As illustrated in FIG. 2S the edges are beveled to relax a sharp corner. FIG. 2S(1) illustrates an implant having a single fillet or bevel 230. The fillet is placed on the implant anterior to the posterior portion of the tibial spine. As shown in FIG. 2S(2) two fillets 230, 231 are provided and used for the posterior chamfer. In FIG. 2S(3) a third fillet 234 is provided to create two cut surfaces for the posterior chamfer.

Turning now to FIG. 2S(4) a tangent of the implant is deselected, leaving three posterior curves. FIG. 2S(5) shows the result of tangent propagation. FIG. 2S(6) illustrates the effect on the design when the bottom curve is selected without tangent propagation. The result of tangent propagation and selection is shown in FIG. 2S(7). As can be seen in FIG. 2S(8-9) the resulting corner has a softer edge but sacrifices less than 0.5 mm of joint space. As will be appreciated by those of skill in the art, additional cutting planes can be added without departing from the scope of the invention.

FIG. 2T illustrates an alternate embodiment of an implant 200 wherein the surface of the tibial plateau 250 is altered to accommodate the implant. As illustrated in FIG. 2T(1-2) the tibial plateau can be altered for only half of the joint surface 251 or for the full surface 252. As illustrate in FIG. 2T(3-4) the posterior-anterior surface can be flat 260 or graded 262. Grading can be either positive or negative relative to the anterior surface. Grading can also be used with respect to the implants of FIG. 2T where the grading either lies within a plane or a body or is angled relative to a plane of the body. Additionally, attachment mechanisms can be provided to anchor the implant to the altered surface. As shown in FIG. 2T(5-7) keels 264 can be provided. The keels 264 can either sit within a plane, e.g. sagittal or coronal plane, or not sit within a plane (as shown in FIG. 2T(7)). FIG. 2T(8) illustrates an implant which covers the entire tibial plateau. The upper surface of these implants are designed to conform to the projected shape of the joint as determined under the steps described with respect to FIG. 1, while the lower surface is designed to be flat, or substantially flat to correspond to the modified surface of the joint.

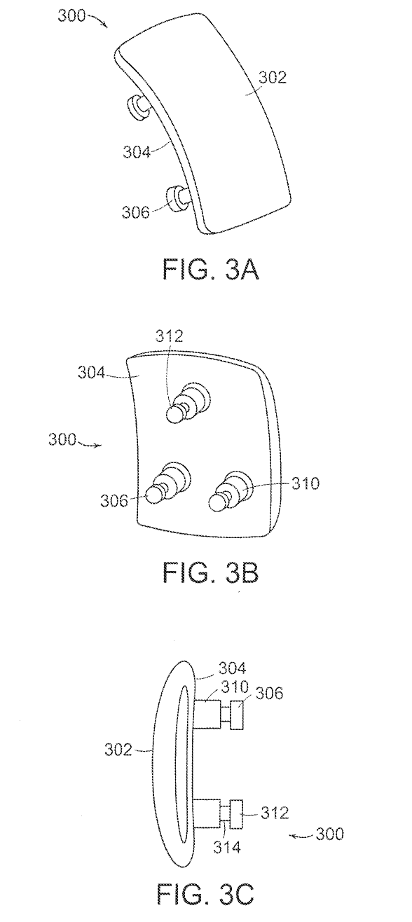

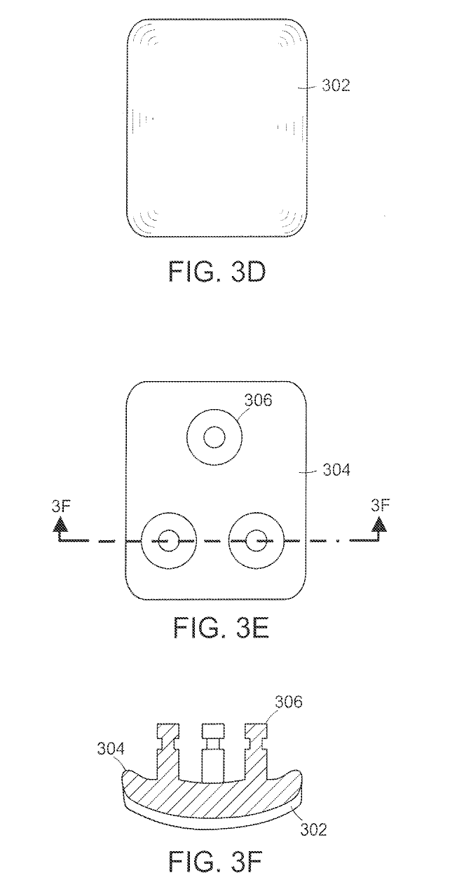

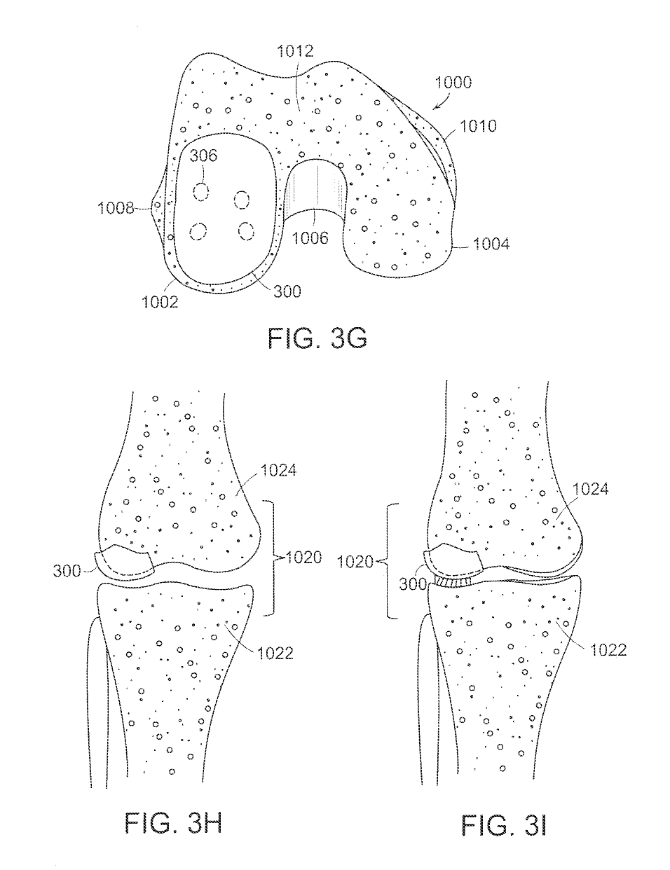

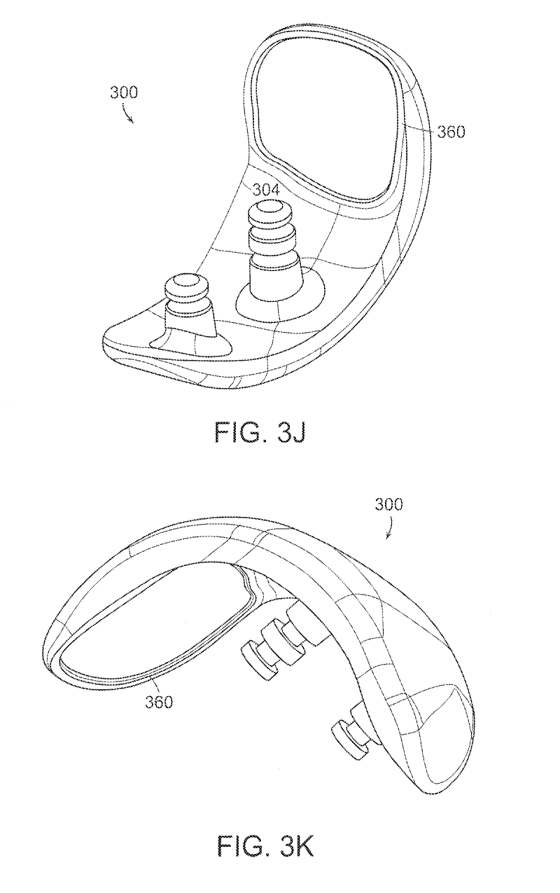

Turning now to FIGS. 3A-I an implant suitable for providing an opposing joint surface to the implant of FIG. 2A is shown. This implant corrects a defect on an inferior surface of the femur 1024 (e.g., the condyle of the femur that mates with the tibial plateau) and can be used alone, i.e., on the femur 1024, or in combination with another joint repair device. Formation of the surfaces of the devices can be achieved using the techniques described above with respect to the implant of FIG. 2.

FIG. 3A shows a perspective view of an implant 300 having a curved mating surface 302 and convex joint abutting surface 304. The joint abutting surface 304 need not form an anatomic or near anatomic fit with the femur in view of the anchors 306 provided to facilitate connection of the implant to the bone. In this instance, the anchors 306 are shown as pegs having notched heads. The notches facilitate the anchoring process within the bone. However, pegs without notches can be used as well as pegs with other configurations that facilitate the anchoring process or cruciate stems. Pegs and other portions of the implant can be porous coated. The implant can be inserted without bone cement or with use of bone cement. The implant can be designed to abut the subchondral bone, i.e. it can substantially follow the contour of the subchondral bone. This has the advantage that no bone needs to be removed other than for the placement of the peg holes thereby significantly preserving bone stock.

The anchors 306 could take on a variety of other forms without departing from the scope of the invention while still accomplishing the same objective of providing increased stability of the implant 300 in the joint. These forms include, but are not limited to, pins, bulbs, balls, teeth, etc. Additionally, one or more joint anchors 306 can be provided as desired. As illustrated in FIG. 3, three pins are used to anchor the implant 300. However, more or fewer joint anchors, cruciate stems, or pins, can be used without departing from the scope of the invention.

FIG. 3B shows a slightly perspective superior view of the bone mating surface 304 further illustrating the use of three anchors 306 to anchor the implant to the bone. Each anchor 306 has a stem 310 with a head 312 on top. As shown in FIG. 3C, the stem 310 has parallel walls such that it forms a tube or cylinder that extends from the bone mating surface 304. A section of the stem forms a narrowed neck 314 proximal to the head 312. As will be appreciated by those of skill in the art, the walls need not be parallel, but rather can be sloped to be shaped like a cone. Additionally, the neck 314 need not be present, nor the head 312. As discussed above, other configurations suitable for anchoring can be used without departing from the scope of the invention.

Turning now to FIG. 3D, a view of the tibial plateau mating surface 302 of the implant 300 is illustrated. As is apparent from this view, the surface is curved such that it is convex or substantially convex in order to mate with the concave surface of the plateau. FIG. 3E illustrates the upper surface 304 of the implant 300 further illustrating the use of three pegs 306 for anchoring the implant 300 to the bone. As illustrated, the three pegs 306 are positioned to form a triangle. However, as will be appreciated by those of skill in the art, one or more pegs can be used, and the orientation of the pegs 306 to one another can be as shown, or any other suitable orientation that enables the desired anchoring. FIG. 3F illustrated a cross section of the implant 300 taken along the lines F-F shown in FIG. 3E. Typically the pegs are oriented on the surface of the implant so that the peg is perpendicular to the femoral condyle, which may not result in the peg being perpendicular to the surface of the implant.

FIG. 3G illustrates the axial view of the femur 1000 having a lateral condyle 1002 and a medial condyle 1004. The intercondylar fossa is also shown 1006 along with the lateral epicondyle 1008 and medial epicondyle 1010. Also shown is the patellar surface of the femur 1012. The implant 300 illustrated in FIG. 3A, is illustrated covering a portion of the lateral condyle. The pegs 306 are also shown that facilitate anchoring the implant 300 to the condyle.

FIG. 3H illustrates a knee joint 1020 from an anterior perspective. The implant 300 is implanted over a condyle. As shown in FIG. 3I the implant 300 is positioned such that it communicates with an implant 200 designed to correct a defect in the tibial plateau, such as those shown in FIG. 2.

FIGS. 3J-K illustrate an implant 300 for placement on a condyle. In this embodiment, at least one flat surface or chamfer cut 360 is provided to mate with a cut made on the surface of the condyle in preparing the joint. The flat surface 360 typically does not encompass the entire proximal surface 304 of the implant 300.

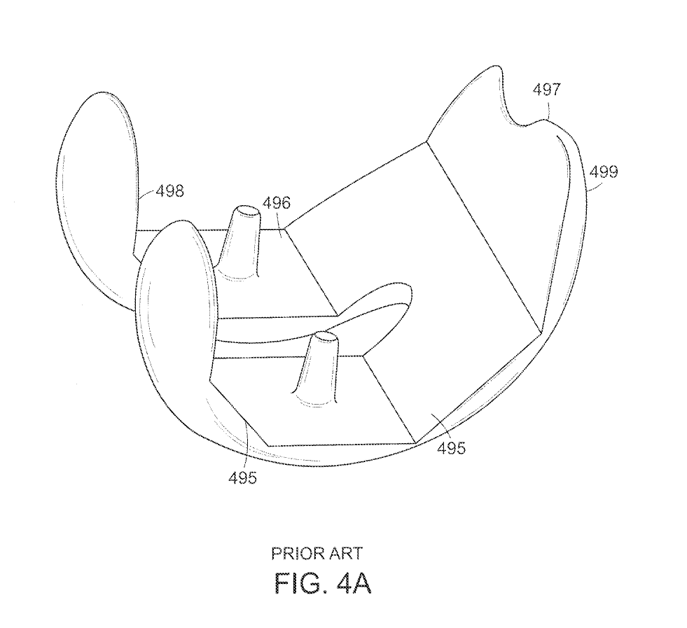

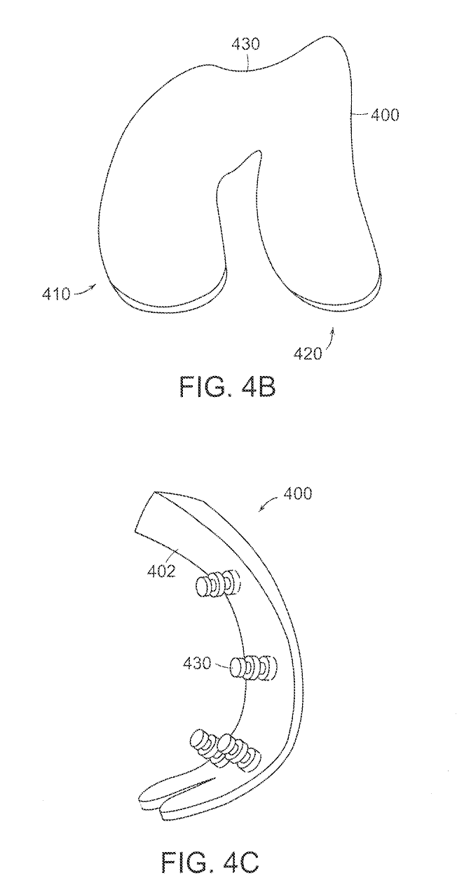

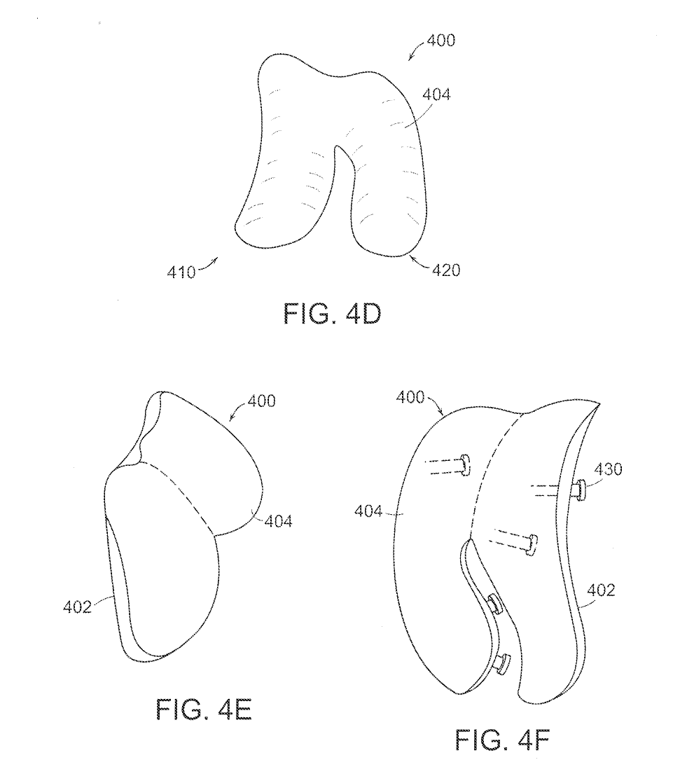

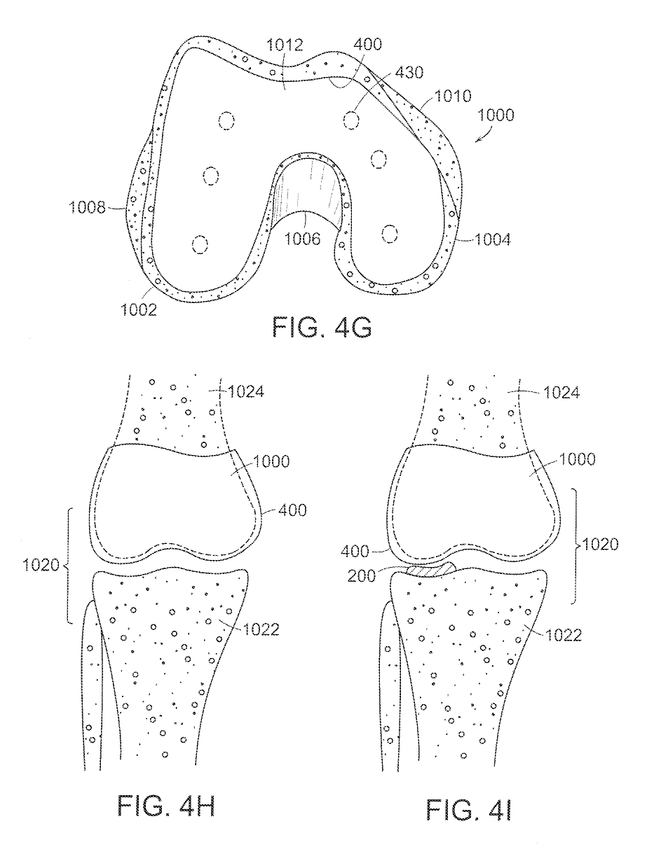

FIG. 4A illustrates the design of a typical total knee arthroplasty (“TKA”) primary knee 499. Posterior cuts 498, anterior cuts 497 and distal cuts 496 are provided as well as chamfer cuts 495.

FIGS. 4B and 4C illustrate another implant 400. As shown in FIG. 4B, the implant 400 is configured such that it covers both the lateral and medial femoral condyle along with the patellar surface of the femur 1012. The implant 400 has a lateral condyle component 410 and a medial condyle component 420 and a bridge 430 that connects the lateral condyle component 410 to the medial condyle component 420 while covering at least a portion of the patellar surface of the femur 1012. The implant 400 can optionally oppose one or more implants, such as those shown in FIG. 2, if desired. FIG. 4C is a side view of the implant of FIG. 4B. AS shown in FIG. 4C, the superior surface 402 of the implant 400 is curved to correspond to the curvature of the femoral condyles. The curvature can be configured such that it corresponds to the actual curvature of one or both of the existing femoral condyles, or to the curvature of one or both of the femoral condyles after resurfacing of the joint. One or more pegs 430 can be provided to assist in anchoring the implant to the bone. As will be appreciated by those of skill in the art, the implant can be configured such that the superior surface contacting a first condyle is configured to male with the existing condyle while a surface contacting a second condyle has one or more flat surfaces to mate with a condyle surface that has been modified.

FIG. 4D illustrates a top view of the implant 400 shown in FIG. 4B. As is should be appreciated from this view, the inferior surface 404 of the implant 400 is configured to conform to the shape of the femoral condyles, e.g. the shape healthy femoral condyles would present to the tibial surface in a non-damaged joint.

FIGS. 4E and F illustrate perspective views of the implant from the inferior surface (i.e., tibial plateau mating surface).

FIG. 4C illustrates the axial view of the femur 1000 having a lateral condyle 1002 and a medial condyle 1004. The intercondylar fossa is also shown 1006 along with the lateral epicondyle 1008. The implant 400 illustrated in FIG. 4B, is illustrated covering both condyles and the patellar surface of the femur 1012. The pegs 430 are also shown that facilitate anchoring the implant 400 to the condyle.

FIG. 4H illustrates a knee joint 1050 from an anterior perspective. The implant 400 is implanted over both condyles. As shown in FIG. 4I the implant 400 is positioned such that it communicates with an implant 200 designed to correct a defect in the tibial plateau, such as those shown in FIG. 2.

As will be appreciated by those of skill in the art, the implant 400 can be manufactured from a material that has memory such that the implant can be configured to snap-fit over the condyle. Alternatively, it can be shaped such that it conforms to the surface without the need of a snap-fit.

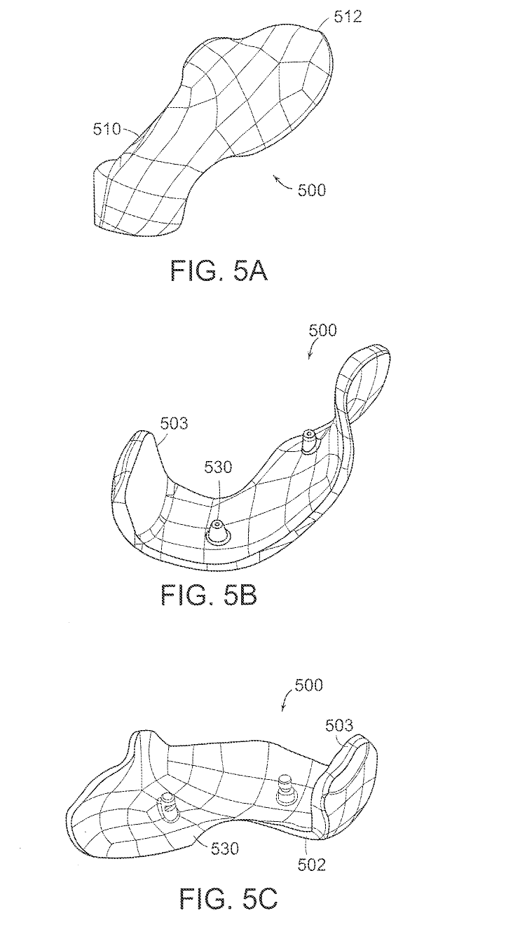

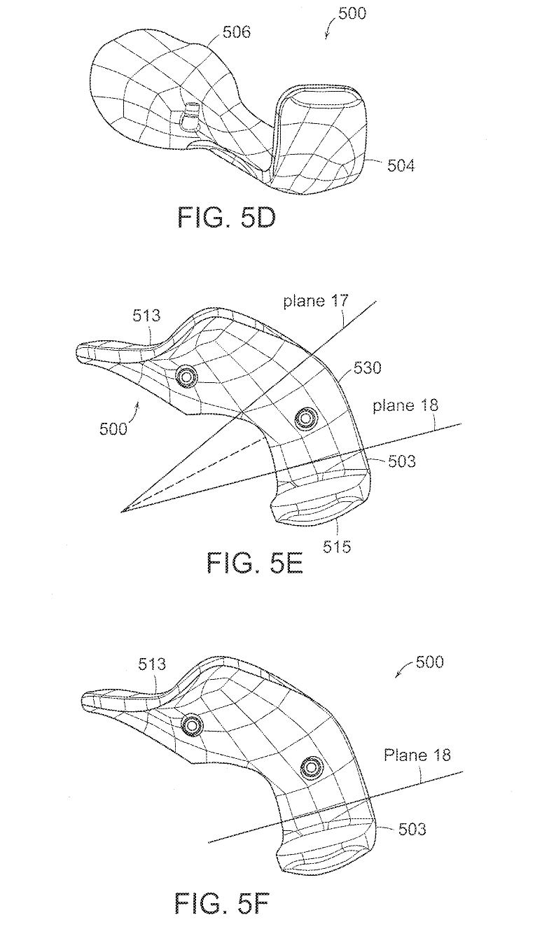

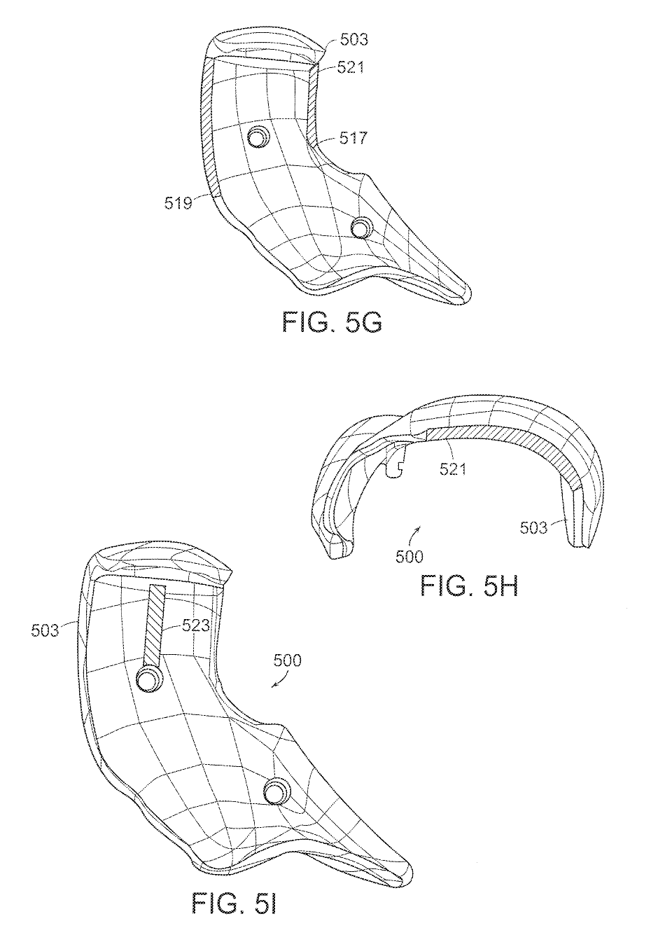

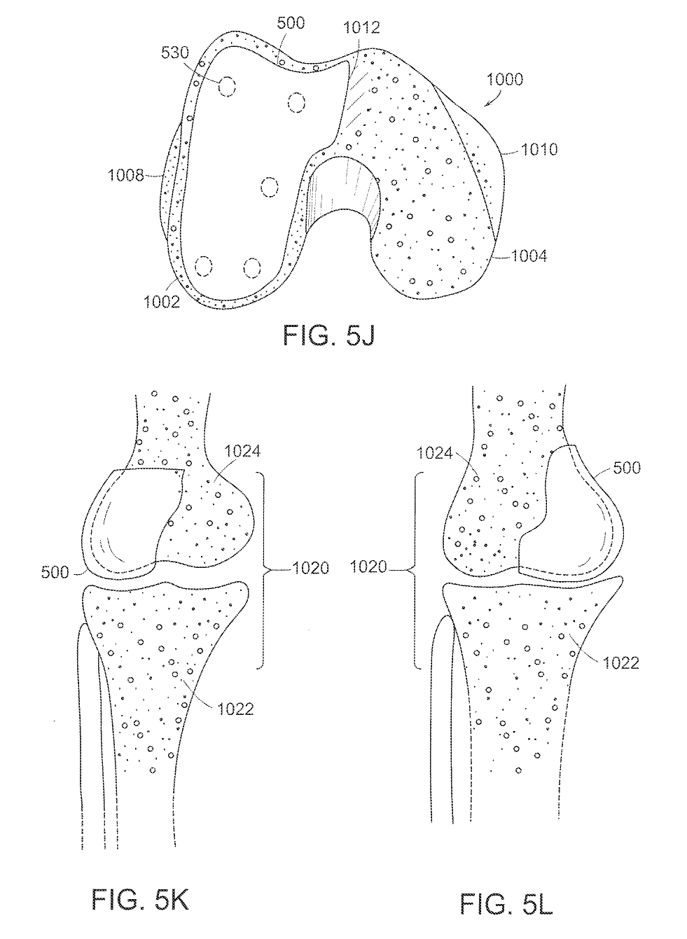

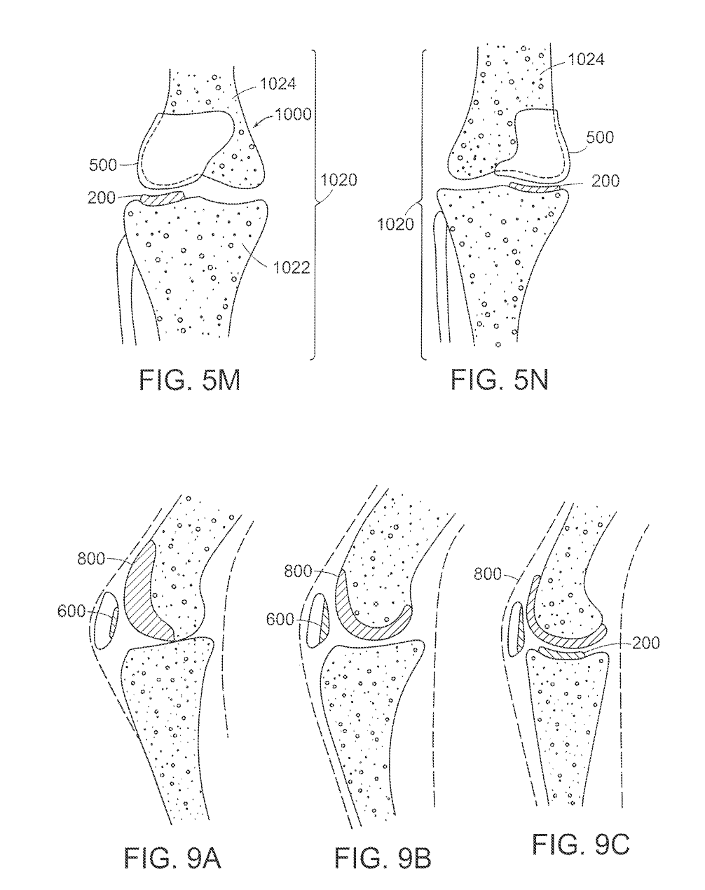

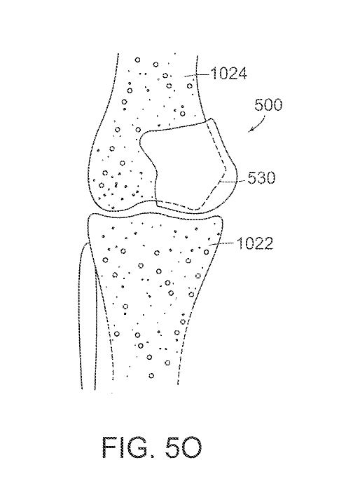

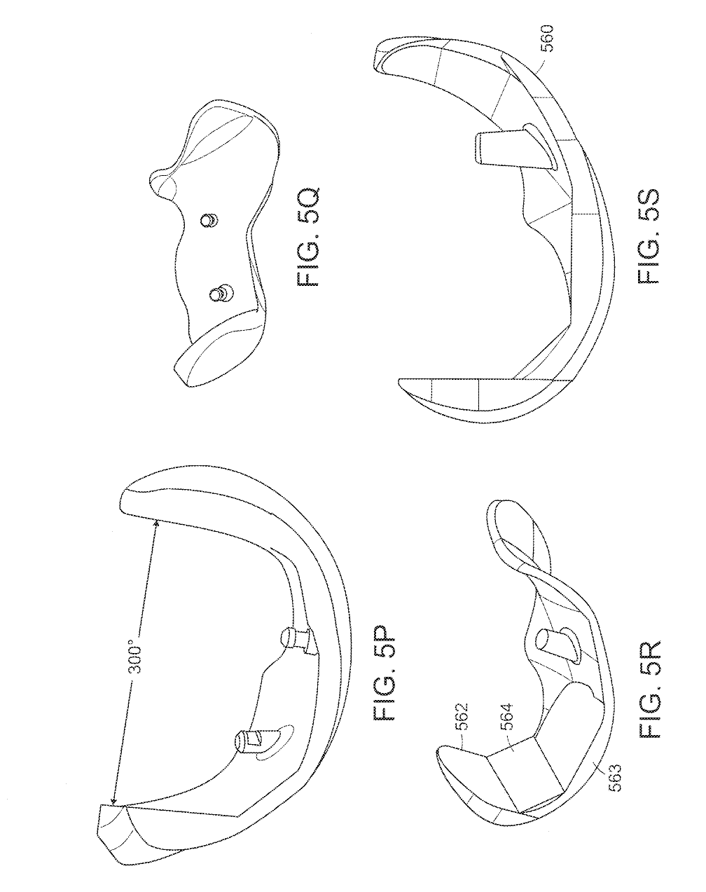

FIGS. 5A and 5B illustrate yet another implant 500 suitable for repairing a damaged condyle. As shown in FIG. 5A, the implant 500 is configured such that it covers only one of the lateral or medial femoral condyles 510. The implant differs from the implant of FIG. 3 in that the implant 500 also covers at least a portion of the patellar surface of the femur 512.

Similar to the implant of FIG. 4, the implant can optionally oppose one or more implants or opposing joint surfaces, such as those shown in FIG. 2, and can be combined with other implants, such as the implants of FIG. 3. FIG. 5C is a perspective side view of the implant of FIG. 5A. As shown in FIG. 5C, the superior surface 502 of the implant 500 is curved to correspond to the curvature of the femoral condyle that it mates with and the portion of the patellar surface of the femur that it covers. One or more pegs 530 can be provided to assist in anchoring the implant to the bone. Additionally, an angled surface 503 can be provided on an interior surface 502 of the condyle component that conforms to an optionally provided cut made on the surface of the joint surface with which the implant mates.

FIG. 5D illustrates a perspective top view of the implant 500 shown in FIG. 5A. As is should be appreciated from this view, the inferior surface 504 of the implant 500 is configured to conform to the projected shape of the femoral condyles, e.g. the shape healthy femoral condyles would present to the tibial surface in a non-damaged joint.

FIG. 5E is a view of the implant 500 showing a hatched three point loading support area which extends from a top portion 513 to a line (plane 17) and from a line (plane 18) to a bottom portion 575. Also illustrated are the pegs 530 extending from the superior surface. FIG. 5F illustrates the superior surface of the implant 500 with the pegs 530 extending from the superior surface. FIG. 5F also illustrates the hatched cantilever loading support area, which extends from the line (plane 18) to the top portion 513 of the implant. The loading forces and directions for each support condition are based on physiological load encounters. Table I shows the Physiological Loadings taken from a study by Seth Greenwald

TABLE 1
Physiological Loadings¹

Set-up

| | “1” | “2” | “3” |
|---|---|---|---|
| Flexion Angle | 0° | 60° | 90° |
| (degree) | | | |
| Normal Force N | 2,900 | 3,263 | 3,625 |
| (lbs.) | (652) | (733.5) | (815) |
| Normal Force | Walking | Stair Descent | Stair Ascent |
| Case | (4.0 × BW^{¥}) | (4.5 × BW^{¥}) | (5.0 × BW^{¥}) |

^{¥}Body Weight (BW) taken as a 60 year old male, with 173 cm height for an average body weight of 74 kg (163 lbs).
¹“Tibial Plateau Surface Stress in TKA: A Factor Influencing Polymer Failure Series III - Posterior Stabilized Designs;” Paul D. Postak, B.Sc, Christine S. Heim, B.Sc, A. Seth Greenwald, D. Phil.; Orthopaedic Research Laboratories, The Mt. Sinai Medical Center, Cleveland, Ohio. Presented at the 62^{nd} Annual AAOS Meeting, 1995.

Using the implant 500 described in this application, the three point loading will occur from set-up 1 (2900 N). To replicate a worst case loading scenario, a 75/25 load distribution (75% of 2900 N=2175 N) will be used. The loading will be concentrated on a 6 mm diameter circular area located directly below and normal to the ped on the bearing surface.

Turning to the cantilever loading shown in FIG. 5F, the loading will occur from set-up 3, or 90°, at a 75/25 load distribution (75% of 3625 N=2719 N). As with the above example, the loading will be concentrated on a 6 mm diameter circular area located at the center of the posterior-most portion of the medial condyle normal to the flat cut surface of the posterior condyle.

FIGS. 5G and H illustrate alternate embodiments of the implant 500 having a rail design that provides one or more rails 521 along medial and/or lateral sides of the implant 500. The rail 521 can be positioned so that it extends along a portion of the medial 517 and/or lateral 519 sides before communicating with the angled surface 503. As will be appreciate, a single side rail 521 can be provided without departing from the scope of the invention.

FIG. 5I illustrates another embodiment of an implant 500 having a keel design. A keel 523 (or centrally formed rail) is provided on the superior surface of the implant. In this embodiment, the keel 523 is located on the surface of the implant, but not at the sides. As will be appreciated, the keel can be centered, as shown, substantially centered, or located off-center. An angled surface 503 can be provided to communicate with a modified joint surface. Alternatively, where the joint surface is worn or modified, the cut 503 could be configured to mate with the worn or modified surface.

FIG. 5J illustrates the axial view of the femur 1000 having a lateral condyle 1002 and a medial condyle 1004. The intercondylar fossa is also shown 1006 along with the lateral epicondyle 1008 and the medial epicondyle 1010. The patellar surface of the femur 1012 is also illustrated. The implant 500, illustrated in FIG. 5A, is shown covering the lateral condyle and a portion of the patellar surface of the femur 1012. The pegs 530 are also shown that facilitate anchoring the implant 500 to the condyle and patellar surface.

FIG. 5K illustrates a knee joint 1020 from an anterior perspective. The implant 500 is implanted over the lateral condyle. FIG. 5L illustrates a knee joint 1020 with the implant 500 covering the medial condyle 1004. As illustrated in FIGS. 5K and L the shape of the implant 500 corresponding to the patella surface can take on a variety of curvatures without departing from the scope of the invention.

Turning now to FIGS. 5M and N the implant 500 is positioned such that it communicates with an implant 200 designed to correct a defect in the tibial plateau, such as those shown in FIG. 2.

In another embodiment of the invention, the implant 500 can have a superior surface 502 which substantially conforms to the surface of the condyle but which has at one flat portion corresponding to an oblique cut on the bone as shown in FIG. 50.

Turning now to FIG. 5P-Q an implant 500 is shown from a side view with a 7° difference between the anterior and posterior cuts.

FIG. 5R-S illustrate an implant 500 having a contoured surface 560 for mating with the joint surface and an anterior cut 561 and a posterior cut 562. FIG. 5S shows the same implant 500 from a slightly different angle. FIG. 5T illustrates another implant 500 having a contoured surface 560 for mating with the joint surface and posterior cut 562, a distal cut 563, and a chamfer cut 564. In this embodiment no anterior cut is provided. FIG. 5U illustrates the implant 500 of FIG. 5T from a side perspective. The cuts are typically less than the cut required for a TKA, i.e., typically less than 10 mm. The design of the cuts for this implant allow for a revision surgery to the knee, if required, at a later date.

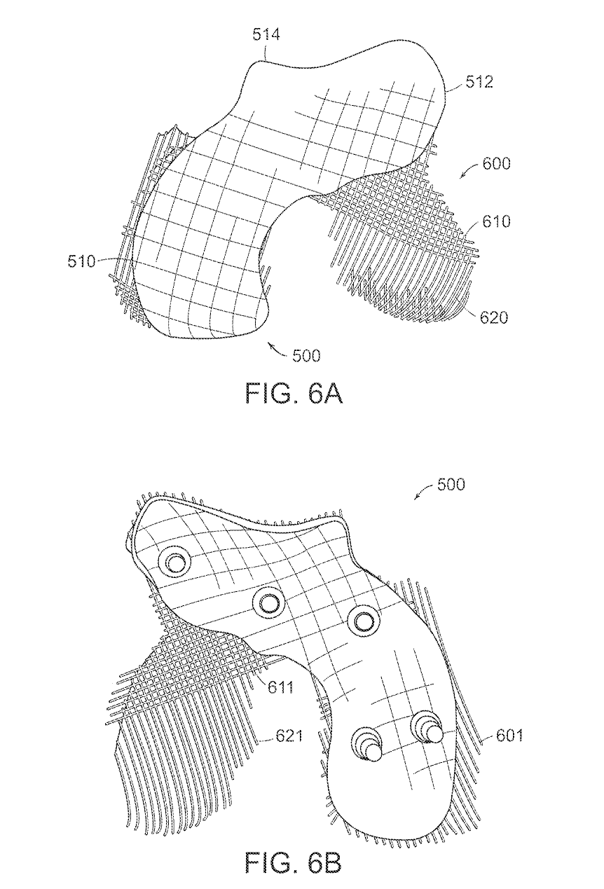

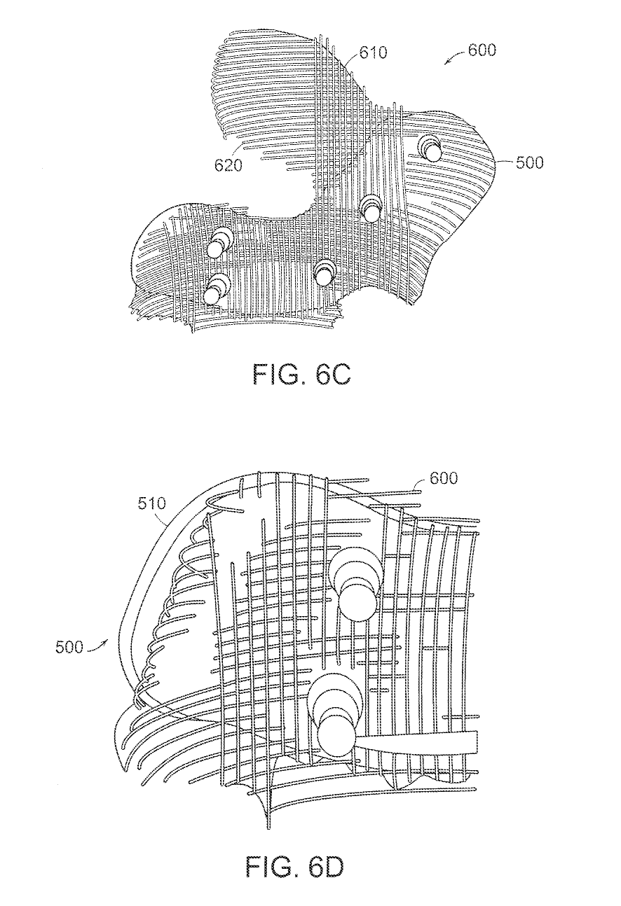

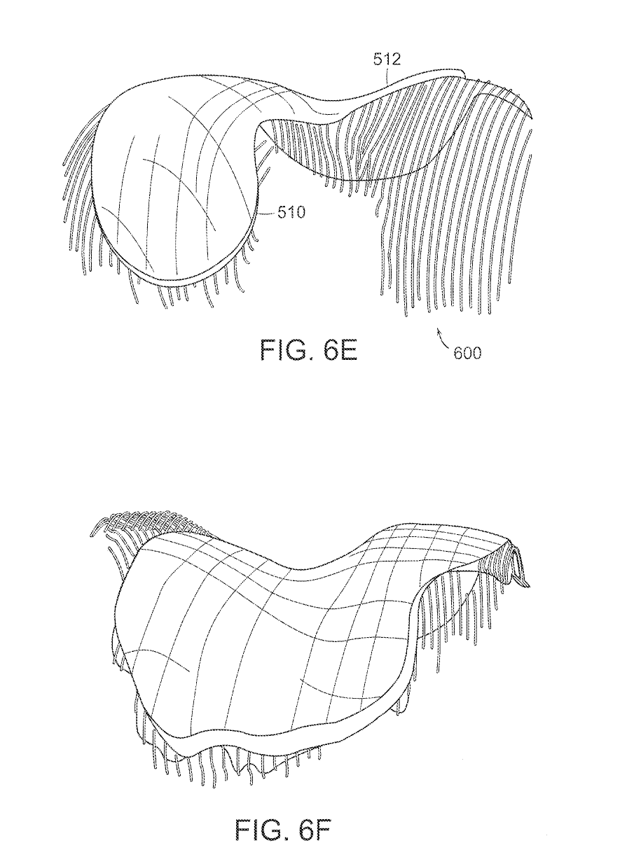

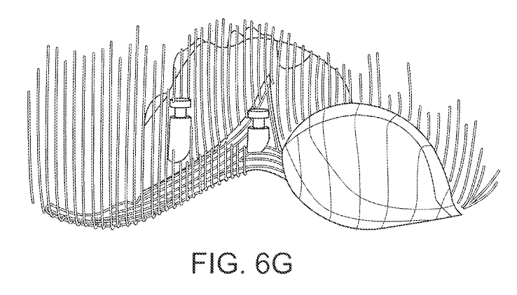

FIGS. 6A-G illustrate the implant 500 of FIG. 5 with a graphical representation of the cross-sections 610, 620 from which a surface shape of the implant is derived. FIG. 6A illustrates a top view of the implant 500 sitting on top of the extracted surface shape 600. This view of the implant 500 illustrates a notch 514 associated with the bridge section of the implant 512 which covers the patellar surface of the femur (or the trochlea region) to provide a mating surface that approximates the cartilage surface. As will be appreciated by those of skill in the art, the shape of an implant designed for the medial condyle would not necessarily be a mirror image of the implant designed for the lateral condyle because of differences in anatomy. Thus, for example, the notch 514 would not be present in an implant designed for the medial condyle and the patellar surface of the femur. Therefore, the implant can be designed to include all or part of the troclea region or to exclude it entirely.

FIG. 6B illustrates a bottom view of the implant 500 layered over another derived surface shape 601. FIG. 6C is a bottom view showing the implant 500 extending through the extracted surface shape 600 shown in FIG. 6A. FIG. 6D is a close-up view of the FIG. 6C showing the condylar wing of the implant covering the extracted surface 600. FIG. 6E illustrates a top posterior view of the implant 500 positioned over the graphical representation of the surface shape 600. FIG. 6F is an anterior view and FIG. 6G is a bottom-posterior view.

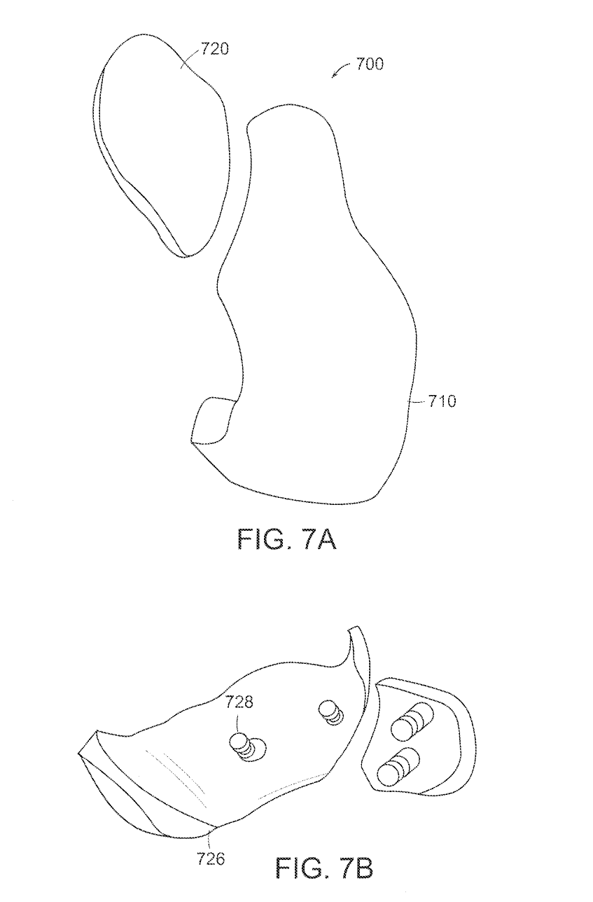

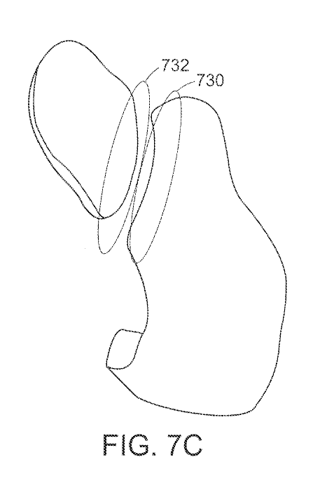

FIG. 7A-C illustrate an implant 700 for correcting a joint similar to the implant 500 above. However, implant 700 consists of two components. The first component 710 engages a condyle of the femur, either medial or lateral depending on the design. The second component 720 engages the patellar surface of the femur. As discussed with the previous embodiments, the surfaces of the implant 700 can be configured such that the distal surface 722 (e.g., the surface that faces the tibial plateau) is shaped based on a projection of the natural shape of the femur compensating the design for valgus or varus deformities and/or flattening of the surface of the femur. Alternatively, the distal surface can be shaped based on the shape of the tibial plateau to provide a surface designed to optimally mate with the tibial plateau. The proximal surface 724 (e.g., the surface that engages the femoral condyle) can be configured such that it mirrors the surface of the femur in either its damaged condition or its modified condition. Likewise, the proximal surface can have one or more flattened sections 726 that form, e.g., chamfer cuts. Additionally the surface can include mechanisms facilitating attachment 728 to the femur, such as keels, teeth, cruciate stems, and the like. The medial facing portion of the condyle implant has a tapered surface 730 while the lateral facing portion of the patellar component also has a tapered surface such that each component presents tapered surfaces 730 to the other component.

By dividing the surfaces of the medial and lateral compartments into independent articulating surfaces, as shown in FIG. 7, the implant provides improved fit of the conformal surfaces to the subchondral bone. Additionally, the lateral-anterior portion of the femur is shielded from stress which could cause bone loss. Also, the smaller size of each component of the implant, enables the implant to be placed within the joint using a smaller incision. Finally, the wear of the patellar component is improved.

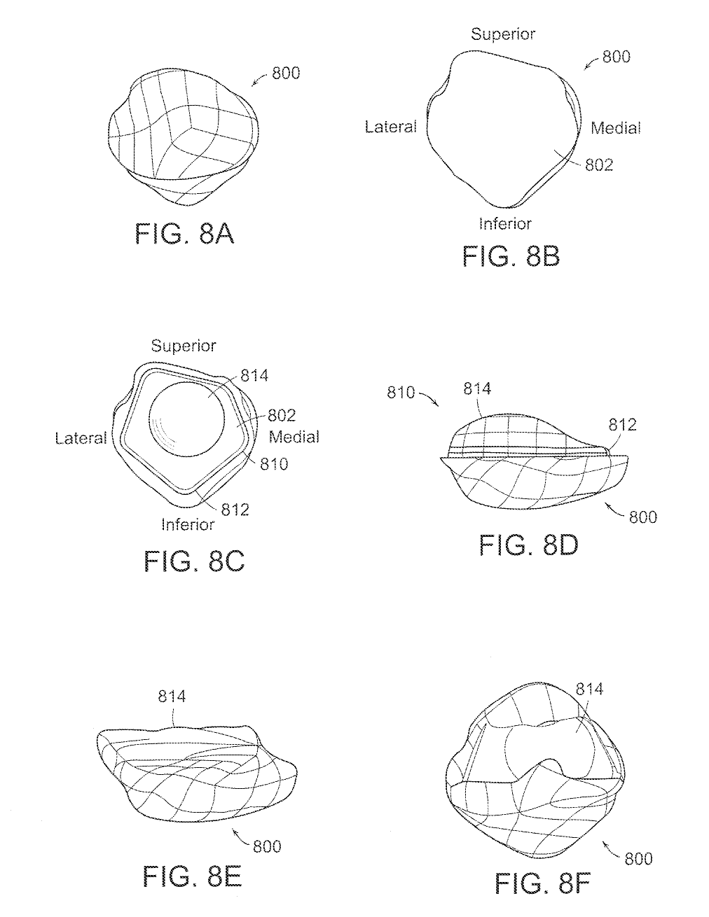

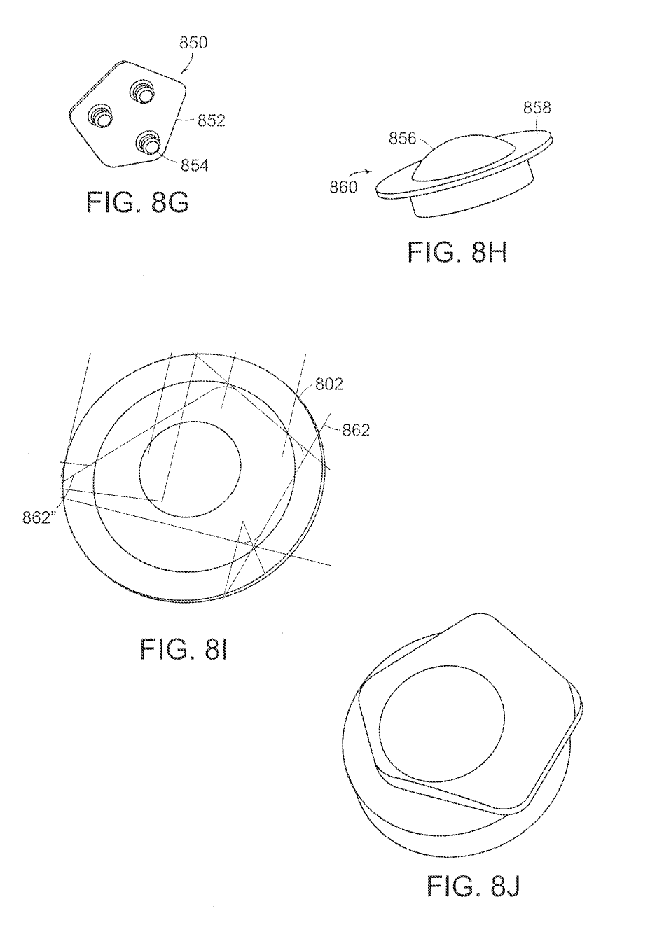

FIGS. 8A-F illustrate a patella 00 with an implants 810. The implant 810 can have one or more pegs, cruciate stems, or other anchoring mechanisms, if desired. As will be appreciated by those of skill in the art, other designs can be arrived at using the teachings of this disclosure without departing from the scope of the invention. FIG. 8A illustrates a perspective view of an intact patella 800. FIG. 8B illustrates the patella 800 wherein one surface of the patella 800 has been cut for form a smooth surface 802 to mate with an implant. FIG. 8C illustrates the patella 800 with an implant 810 positioned on the smooth surface 802. The implant 810 has a plate structure 812 that abuts the smooth surface of the patella 802 and a dome 814 positioned on the plate 812 so that the dome is positioned in situ such that it will match the location of the patellar ridge. The implant 810 can be configured such that the edge of the plate is offset 1 mm from the actual edge of the patella, as illustrated. As will be appreciated by those of skill in the art, the plate 812 and dome 814 can be formed as a single unit or formed from multiple components. FIG. 8D is a side view of the implant 810 positioned on the patella 800. As shown, the dome is positioned on the implant such that it is off-center. Optimal positioning of the dome will be determined by the position of the patellar ridge.

Turning now to FIGS. 8E-F, the implant 810 is shown superimposed on the unaltered patella 800 in order to illustrate that the position of the dome 814 of the implant corresponds to the location of the patellar ridge.

FIGS. 8G-J illustrate an alternative design for the patellar implant. FIG. 8G illustrates the implant 850 in its beginning stages as a blank with a flat inferior surface 852 having pegs 854 extending there from for anchoring to the patella. The articular or superior surface 860 has a rounded dome 856, and a round plate section 858 that can be machined to match the bone cut. The articular surface 860 takes on the appearance of a “hat” or sombrero, having a dome with a rim. The center of the dome 856 is also the center of the bearing surface. The rim 555 is cut to conform to the needs of the particular patient. FIG. 8J illustrates an implant which has been formed from the blank shown in FIGS. 8G-I. FIG. 8I shows a plurality of possible cut lines 862, 862′ for purposes of illustration.

FIGS. 9A-C illustrate a lateral view of a knee 1020 having a combination of the implants of implanted thereof. In FIG. 9A, an implant covering the condyle 900, is illustrated. Suitable implants can be, for example, those shown in FIGS. 3-8, as will be appreciated the portion of the condyle covered anterior to posterior can include the entire weight bearing surface, a portion thereof, or a surface greater than the weight bearing surface. Thus, for example, the implant can be configured to terminate prior to the sulcus terminalis or after the sulcus terminalis (e.g., the groove on the femur that coincides with the area where load bearing on the joint surface stops). As shown in FIGS. 9A-B, a patellar implant 900 can also be provided. FIG. 9C illustrates a knee having a condyle implant 900, a patellar implant 800 and an implant for the tibial plateau 200.

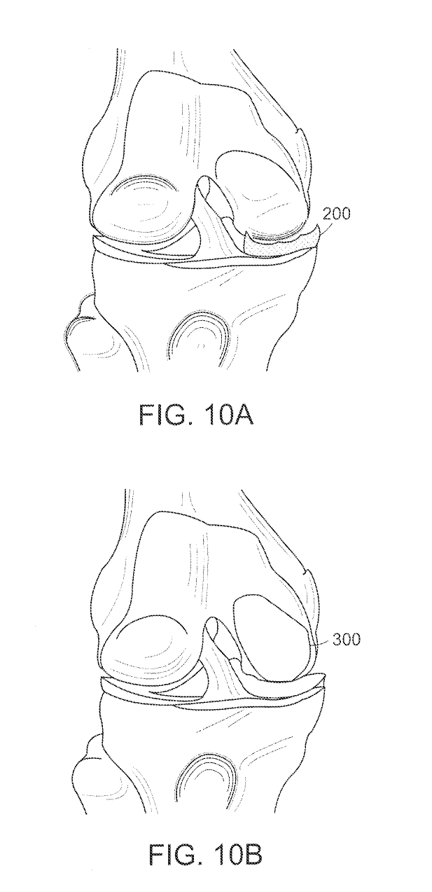

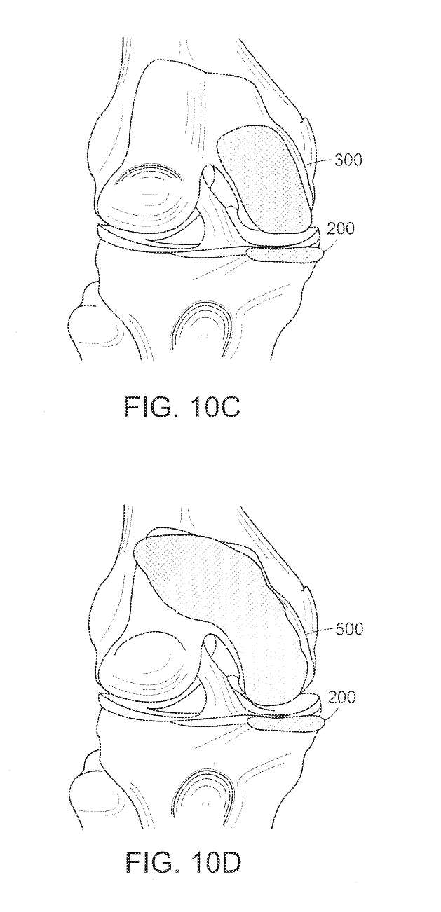

FIGS. 10A-D provide an alternate view of the coronal plane of a knee joint with one or more implants described above implanted therein. FIG. 10A illustrates a knee having a tibial implant 200 placed therein. FIG. 10B illustrates a knee with a condyle implant 300 placed therein. As described above, a plurality of the implants taught herein can be provided within a joint in order to restore joint movement. FIG. 10C illustrates a knee joint having two implants therein. First, a tibial implant 200 is provided on the tibial plateau and a second implant 300 is provided on the facing condyle. As will be appreciated by those of skill in the art. The implants can be installed such that the implants present each other mating surfaces (as illustrated), or not. For example, where the tibial implant 200 is placed in the medial compartment of the knee and the condyle implant 300 is placed in the lateral compartment. Other combinations will be appreciated by those of skill in the art. Turning now to FIG. 10D, a tibial implant 200 is provided along with a bicompartmental condyle implant 500. As discussed above, these implants can be associated with the same compartment of the knee joint, but need not be.

The arthroplasty system can be designed to reflect aspects of the tibial shape, femoral shape and/or patellar shape. Tibial shape and femoral shape can include cartilage, bone or both. Moreover, the shape of the implant can also include portions or all components of other articular structures such as the menisci. The menisci are compressible, in particular during gait or loading. For this reason, the implant can be designed to incorporate aspects of the meniscal shape accounting for compression of the menisci during loading or physical activities. For example, the undersurface 204 of the implant 200 can be designed to match the shape of the tibial plateau including cartilage or bone or both. The superior surface 202 of the implant 200 can be a composite of the articular surface of the tibia (in particular in areas that are not covered by menisci) and the meniscus. Thus, the outer aspects of the device can be a reflection of meniscal height. Accounting for compression, this can be, for example, 20%, 40%, 60% or 80% of uncompressed meniscal height.

Similarly the superior surface 304 of the implant 300 can be designed to match the shape of the femoral condyle including cartilage or bone or both. The inferior surface 302 of the implant 300 can be a composite of the surface of the tibial plateau (in particular in areas that are not covered by menisci) and the meniscus. Thus, at least a portion of the outer aspects of the device can be a reflection of meniscal height. Accounting for compression, this can be, for example, 20%, 40%, 60% or 80% of uncompressed meniscal height. These same properties can be applied to the implants shown in FIGS. 4-8, as well.

In some embodiments, the outer aspect of the device reflecting the meniscal shape can be made of another, preferably compressible material. If a compressible material is selected it is preferably designed to substantially match the compressibility and biomechanical behavior of the meniscus. The entire device can be made of such a material or non-metallic materials in general.

The height and shape of the menisci for any joint surface to be repaired can be measured directly on an imaging test. If portions, or all, of the meniscus are torn, the meniscal height and shape can be derived from measurements of a contralateral joint or using measurements of other articular structures that can provide an estimate on meniscal dimensions.

In another embodiment, the superior face of the implants 300, 400 or 500 can be shaped according to the femur. The shape can preferably be derived from the movement patterns of the femur relative to the tibial plateau thereby accounting for variations in femoral shape and tibiofemoral contact area as the femoral condyle ilexes, extends, rotates, translates and glides on the tibia and menisci.

The movement patterns can be measured using any current or future test know in the art such as fluoroscopy, MRI, gait analysis and combinations thereof.

In various embodiments, a joint implant may include two or more components that are slideably engageable forming a mobile bearing. The mobile bearing can help provide more unconstrained or more physiologic motion in the joint, for example, knee motion of the femur relative to the tibia.

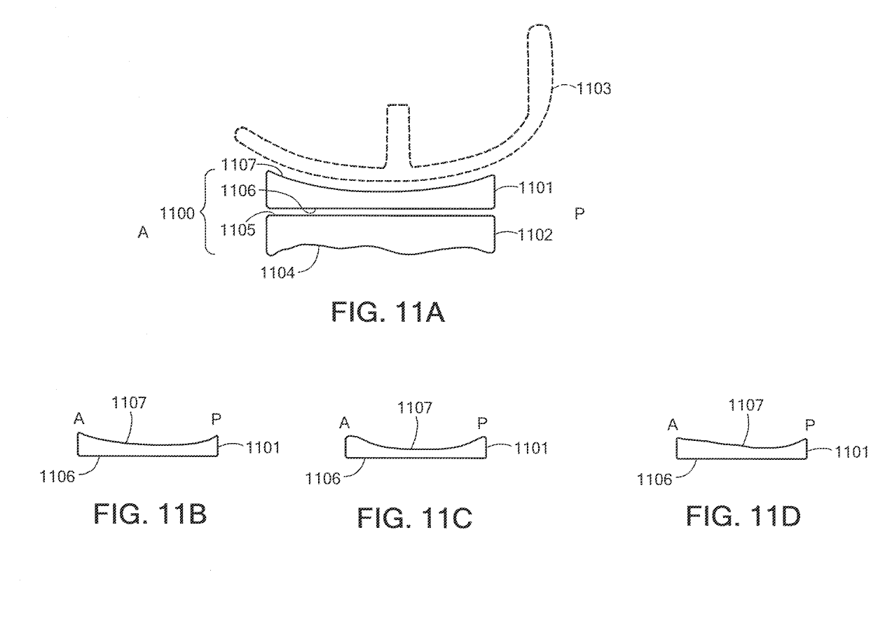

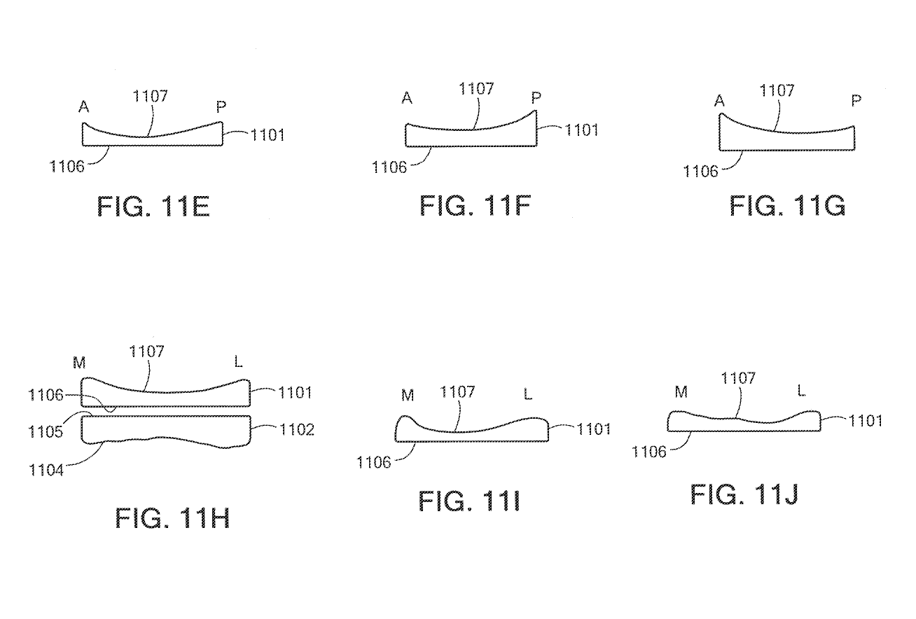

FIG. 11A shows a joint implant 1100 that includes a mobile bearing, in accordance with one embodiment of the invention. The implant 1100 includes a first component 1102 and a bearing component 1101. The first component 1102 includes a bone-facing surface 1104 for engaging bone or cartilage of a joint, and an external surface 1105. The bearing component 1101 includes a first surface 1106 for slidingly engaging the external surface of the first component 1101, and a second surface 1107 for articulation with a second component 1103 and/or other bone or cartilage surface.

The bone facing surface 1104 may be a mirror image of, and engage, a substantially uncut articular cartilage surface and/or a substantially uncut subchondral bone surface. The bone-facing surface may be formed using, without limitation, the above-described imaging and modeling techniques. The bone facing surface 1104 may match and conform with the existing underlying surface to achieve an anatomic or near anatomic fit, such that it replicates the natural joint anatomy. Such a non-invasive approach advantageously does not require surgical resection of the articular bone surface.

In various embodiments, the joint implant 1100 may be configured to be used, without limitation, in a hip, knee, ankle, should, elbow, wrist, or hand. For example, the joint implant 1100 maybe directed at a knee, with the bone facing surface 1104 of the first component 1102 engaging a tibial articular surface, and the second surface 1107 of the bearing component 1101 articulating with a femoral component 1103.

The bearing surface between the bearing component 1101 and the first component 1102 may be, without limitation, flat, as shown in FIG. 11A. The second surface of the bearing component 1101, which may, for example, face the femur in a knee implant (or, for example, face an implant 1103 covering a portion of the femoral condyle), may have a constant or variable radii both in anteroposterior and/or mediolateral directions as shown in FIGS. 11B-J, in accordance with various embodiments of the invention. For example, the second surface 1107 of the bearing component 1101 in a knee implant may include one or more concavities and/or convexities so as to match the superior surface of the replaced meniscal cartilage and/or provide for proper articulation with a femoral implant and/or articular surface.

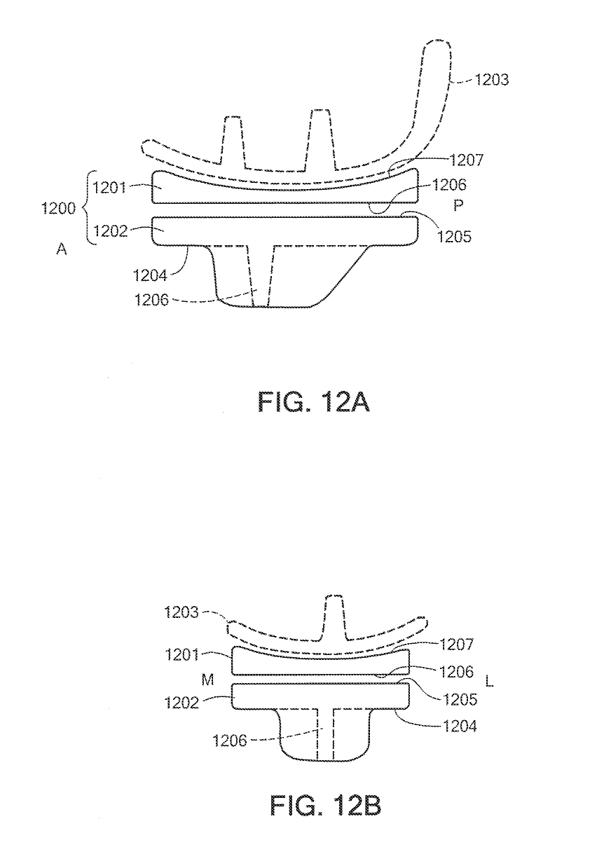

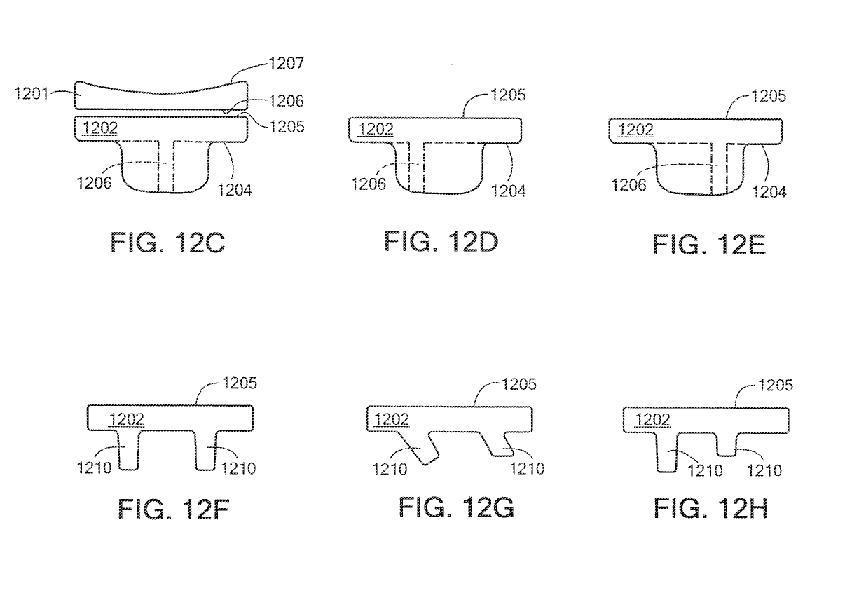

FIGS. 12A-E show a joint implant 1200 having a mobile bearing that can be fixedly anchored into the articular joint, such as, in a knee implant, the tibial plateau (not shown), in accordance with various embodiments of the invention. For example, one or more fins 1205 and 1206 positioned on the bone facing surface of the first component 1202 of the joint implant 1200 maybe used to anchor the joint implant 1200 into the tibial plateau. The fins 1205 and 1206 may be perpendicular relative to each other or they may be oriented at an angle other than 90 degrees. A transverse fin 1206 may be located in the center of an anteroposterior fin 1205, as shown in FIG. 12B. In other embodiments, the transverse fin 1206 may be located anterior or posterior relative to the center of the anteroposterior fin, as shown in FIGS. 12D-E.

Alternatively, the joint component 1200 may be anchored via one or more pegs 1210 into, without limitation, the tibial plateau, as shown in FIGS. 12F-H, in accordance with various embodiments of the invention. These pegs 1210 may be perpendicular to the articular surface, as shown in FIG. 12F or at an angle other than 90 degrees to the articular surface, as shown in FIG. 12G. The pegs may have the same length as shown in FIG. 12F, or a different length, as shown in FIG. 2H. With an anterior incision, a shorter posterior peg may advantageously allow a more minimally invasive approach by reducing the size of the incision.

Any anchoring mechanism known in the art may be used.

The bone-facing surface of the first component 1202 of the joint implant 1200 may sit on top of, and conform with, the subchondral bone and/or articular cartilage without cutting the tibia, with only the anchoring mechanism protruding into the bone. Alternatively, the surgeon may cut the articular surface (e.g., the tibial plateau in a knee implant), and the implant can be seated on top of the cut. Standard techniques for cutting the articular surface known in the art may be used. Note that in various embodiments, the bone-facing surface may conform with the subchondral bone and/or articular cartilage such that the first component is sufficiently self-centering, with requiring any anchoring mechanism or cuts.

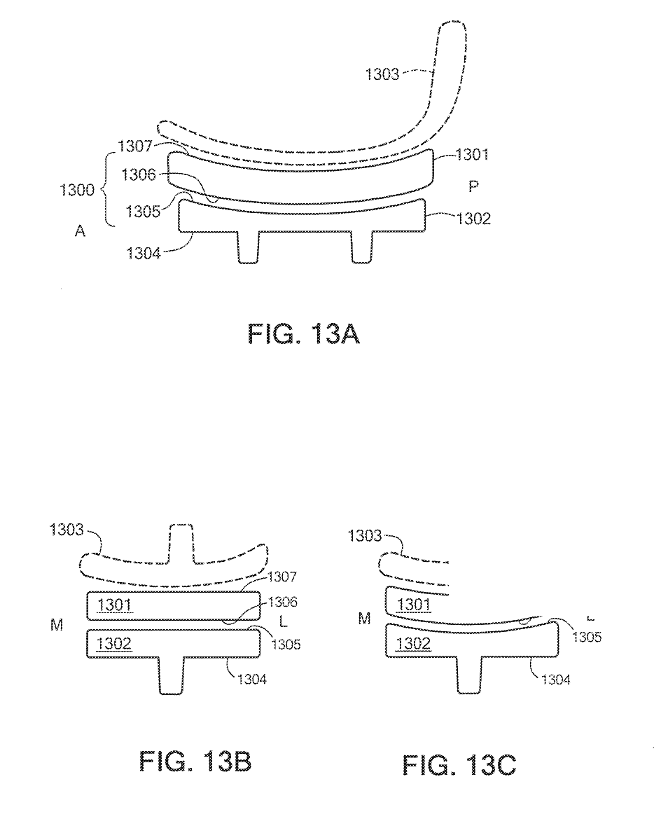

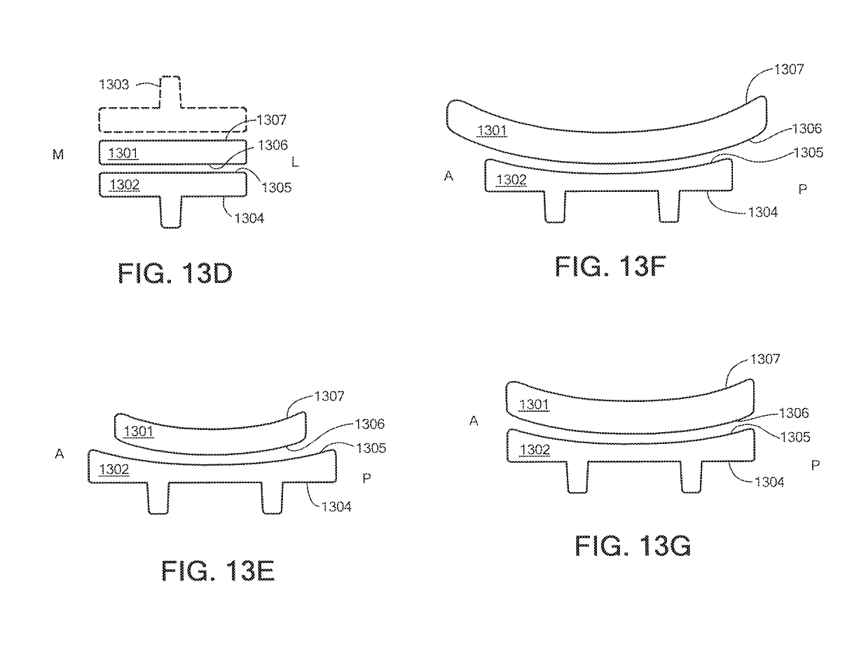

FIG. 13A shows a joint implant 1300 having a bearing surface between the bearing component 1301 and the first component 1302 that is curved rather than flat, in accordance with one embodiment of the invention. For example, the external surface 1305 of the first component 1302 may be concave, rising towards the bearing component 1301 upwardly on one or more sides. The second surface 1307 of the bearing component 1301 (e.g., that faces the femur in a knee implant) may have a constant or variable radii both in anteroposterior and/or mediolateral direction. A variable radius may advantageously accommodate different femoral radii during flexion and translation of the condyle.

In various embodiments, the bearing surface between the bearing component 1301 and the first component 1302 may be flat and the second surface 1307 of the bearing component 1301 (e.g., facing the femoral component in a knee implant) may also be flat, as shown in FIG. 13B. In other embodiments, the bearing surface between the bearing component 1301 and first component 1302 may be curved and the second surface 1307 of the bearing component 1301 may also be curved, as shown in FIG. 13C. In still other embodiments, the bearing surface between the bearing component 1301 and the first component 1302 may be flat, with the second surface 1307 of the bearing component 1301 flat, and the femoral component 1303 flat in one dimension (preferably the coronal dimension in a knee implant), as shown in FIG. 13D.

In another embodiment, the bearing component 1301 of the joint implant 1300 may be smaller in one or more dimensions than the first component 1302, as shown in FIG. 13E. In other embodiments, the bearing component 1301 may be longer in one or more dimensions than the first component 1302, as shown in FIG. 13F, or it can have substantially the same length or width or both than the first component 1302, as shown in FIG. 3G.

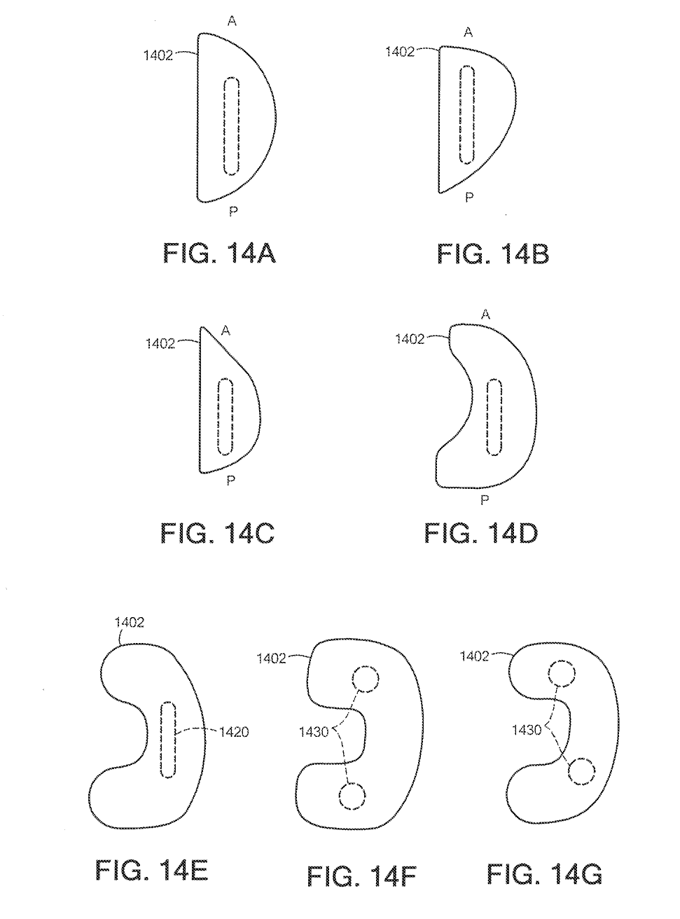

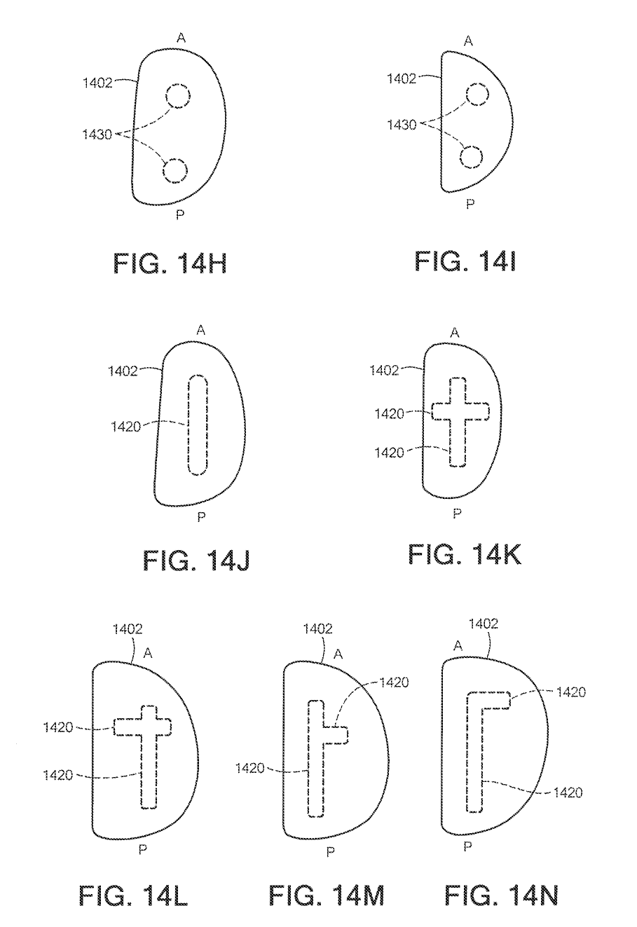

FIGS. 14A-N shows a top view of perimeter, keel and peg configurations for the first component 1402 of a joint implant that includes a mobile bearing, in accordance with various embodiments of the invention. FIG. 14A shows the first component 1402 of a joint implant, which illustratively is a tibial implant, having a perimeter with a substantially constant radius, in accordance with one embodiment of the invention. In other embodiments, the first component 1402 may have a variable radius, as shown in FIG. 14B. FIG. 14C shows an example of a first component 1402 with a variable radius and a substantially straight surface oriented towards the intercondylar notch area. In other embodiments, the surface oriented towards the intercondylar notch may be concave or convex in at least one portion, as shown in FIG. 14D. In FIG. 14D, the concavity helps avoid the tibial spines intraoperatively, with the outer perimeter of the first component being kidney shaped, as shown in FIGS. 14D-G. FIGS. 14E-G demonstrate various embodiments with concave and convex tibial component perimeters and various keel 1420 (FIG. 14E) and peg 1430 (FIGS. 4F-G) positions.

FIG. 14H shows a first component 1402 that is substantially round in perimeter using two exemplary pegs 1430 for attachment, in accordance with one embodiment of the invention. FIG. 14I shows an exemplary semilunar shaped first component 1402 using two exemplary pegs 1430 for attachment. The outer perimeter of the first component 1402 may be substantially round (FIG. 14H) or can include more pointed aspects (FIG. 14I).

FIG. 14J shows a first component 1402 with a single fin 1420 for anchoring, in accordance with one embodiment of the invention. A first component 1402 with two perpendicularly arranged keels 1420 for anchoring is shown in FIG. 14K, in accordance with another embodiment of the invention.

FIG. 14L shows a first component 1402 with the fin 1420 located in substantially the coronal plane moved more anteriorly relative to the fin 1420 located in substantially the sagittal plane, in accordance with one embodiment of the invention. FIGS. 14M-N show other examples of potential configurations of the keels 1420.

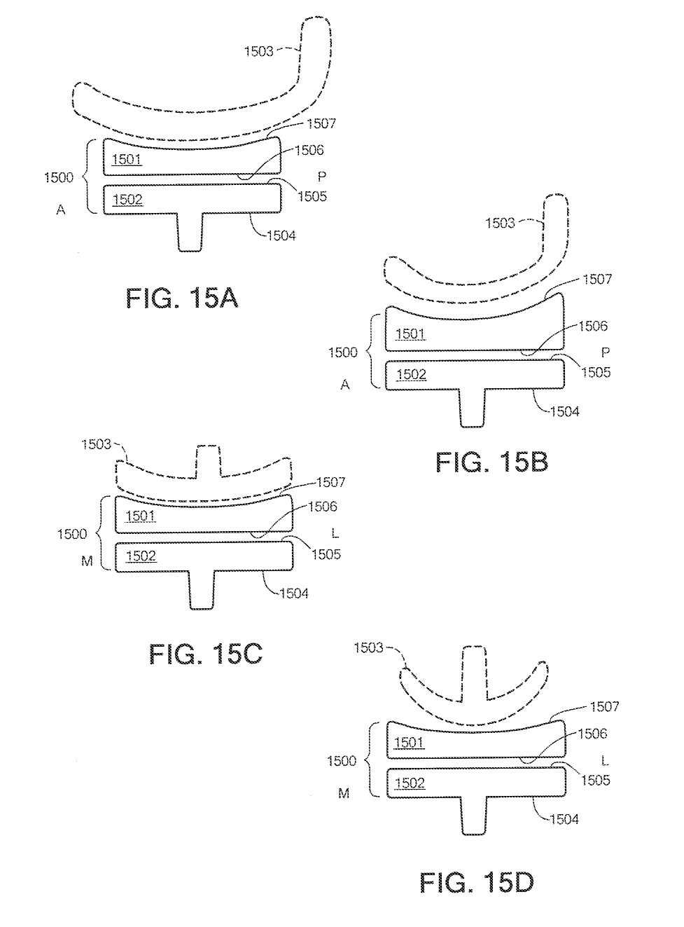

FIG. 15A shows a joint implant 1500 with the second surface 1507 of the bearing component 1501 having a constant radius in the sagittal plane, in accordance with one embodiment of the invention. FIG. 15B shows a joint implant 1500 with the second surface 1507 of the bearing component 1501 having a variable radius in the sagittal plane. FIG. 15C shows a joint implant 1500 with the second surface 1507 of the bearing component 1501 having a constant radius substantially matching that of, for example, the femoral condyle (or, for example, matching an implant 1503 covering a portion of the femoral condyle) in the coronal plane. FIG. 15D shows a joint implant 1500 with the second surface 1507 of the bearing component 1501 having a constant radius not substantially matching, for example, that of the femoral condyle (or, for example, not matching an implant 1503 covering a portion of the femoral condyle) in the coronal plane.

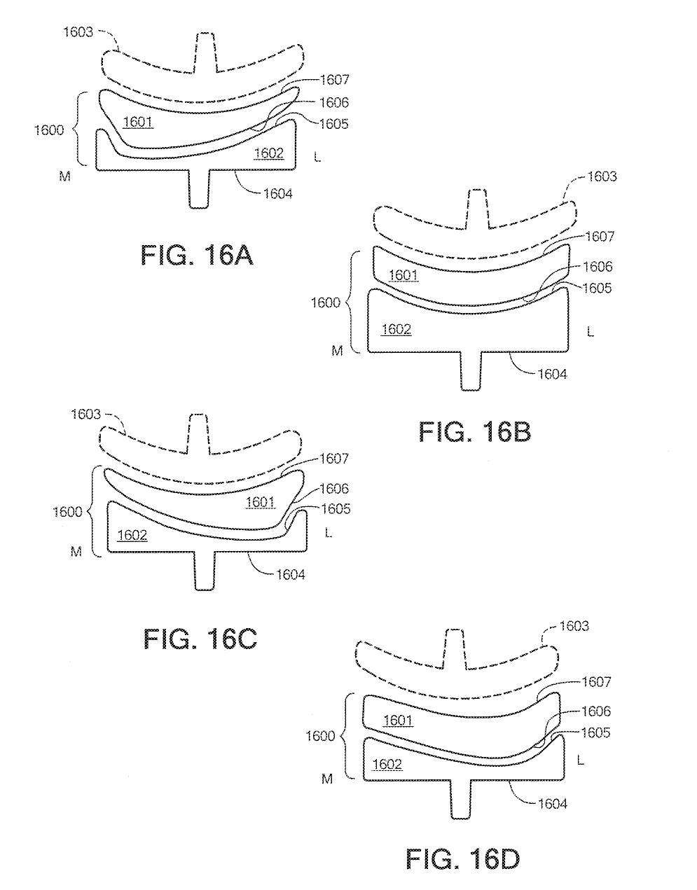

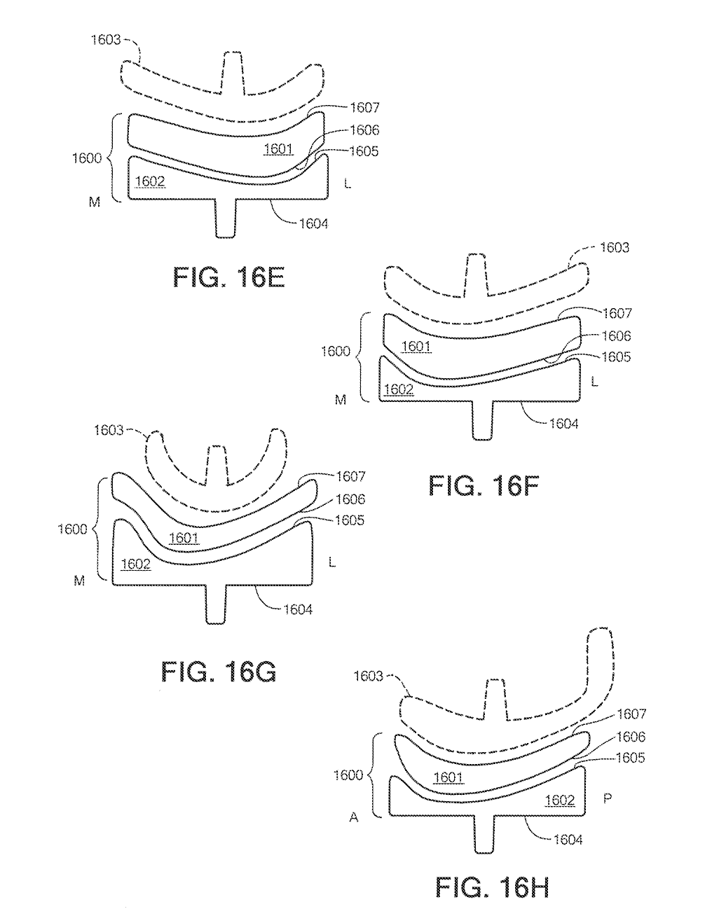

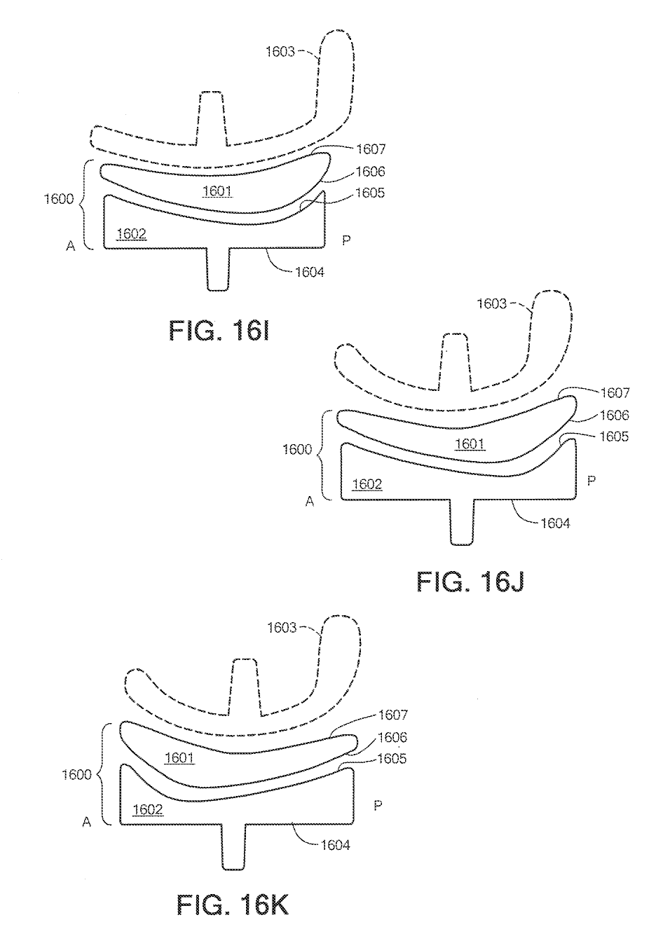

FIG. 16A shows a joint implant 1600 with a bearing surface between the bearing component 1601 and the first component 1602 that is asymmetrical, with varying curvature radii, in accordance with one embodiment of the invention. Radii may vary in one or more dimensions. Radii may be chosen such that the external surface 1605 of the first component 1602 and the first surface 1606 of the bearing component 1601 simulate near physiologic motion of the components, for example matching, in a knee implant, tibiofemoral rotation and translation. The bearing surface may include, without limitation, one or more convexities and/or concavities. The second surface 1607 of the bearing component 1601 facing, in a knee implant for example, the femoral condyle has, without limitation, a substantially constant radius.

FIG. 16B shows a joint implant 1600 with a bearing surface between the bearing component 1601 and the first component 1602 that is symmetrical, with constant radii, in accordance with one embodiment of the invention. Radii may be constant in only one dimension or more.

FIG. 16C shows a joint implant 1600 with a bearing surface between the bearing component 1601 and the first component 1602 that is asymmetrical, with varying radii. The second surface 1607 of the bearing component 1601 facing, in a knee implant for example, the femoral condyle has, without limitation, a substantially constant radius.

FIGS. 16D-E demonstrate joint implants 1600 with a bearing surface between the bearing component 1601 and the first component 1602 that is asymmetrical, with varying radii, and the second surface 1607 of the bearing component 1601 that has also varying radii, in accordance with various embodiments of the invention. In FIG. 16D, the second component (e.g. the femoral component in a knee implant) 1603 has a substantially constant radius. In FIG. 16E, the second component 1603 has a variable radius.

FIG. 16F shows a joint implant 1600 having a second surface 1607 of the bearing component 1601 with variable radii that are, however, substantially matching the radii of the second component 1603, in accordance with one embodiment of the invention. FIG. 16G shows a joint implant 1600 having a second surface 1607 of the bearing component 1601 with variable radii that are, however, substantially not matching the radii of the second component 1603, in accordance with one embodiment of the invention.

FIG. 16H shows a joint device 1600, such as a tibial implant, in the sagittal plane with the bearing surface of the bearing component 1601 and the first component 1602 having variable radii in the sagittal plane, and the second surface 1607 of the bearing component 1601 facing the second component 1603 (e.g., the femoral condyle) having a substantially constant radius, in accordance with one embodiment of the invention. The smallest radii are observed anteriorly in the bearing surface.

FIG. 16I shows a joint device 1600, such as a tibial implant, in the sagittal plane with the bearing surface of the two components 1601 and 1602 having variable radii in the sagittal plane, and the second surface 1607 of the bearing component 1601 facing the second component 1603 (e.g., the femoral condyle) having a substantially constant radius, in accordance with one embodiment of the invention. The smallest radii are observed posteriorly in the bearing surface.

FIGS. 16J-K show implants in the sagittal plane with the bearing surface of the two components 1601 and 1602 having variable radii in the sagittal plane, and the second surface 1607 of the bearing component 1601 facing the second component 1603 (e.g., the femoral condyle) having also variable radii, in accordance with various embodiments of the invention. In FIG. 16J, the smaller radii of the top surface facing the second component 1603 are seen centrally to posteriorly. In FIG. 16 K, the smaller radii of the top surface facing the second component 1603 are seen anteriorly.

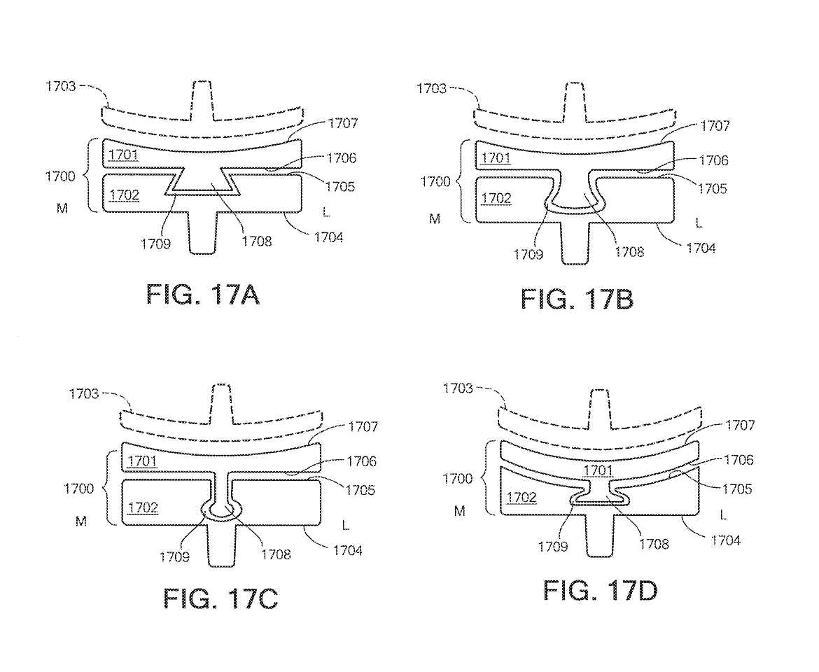

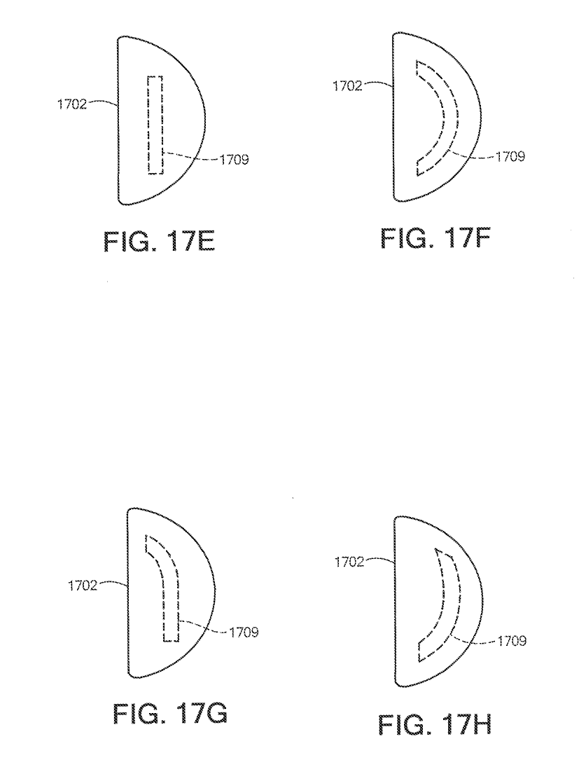

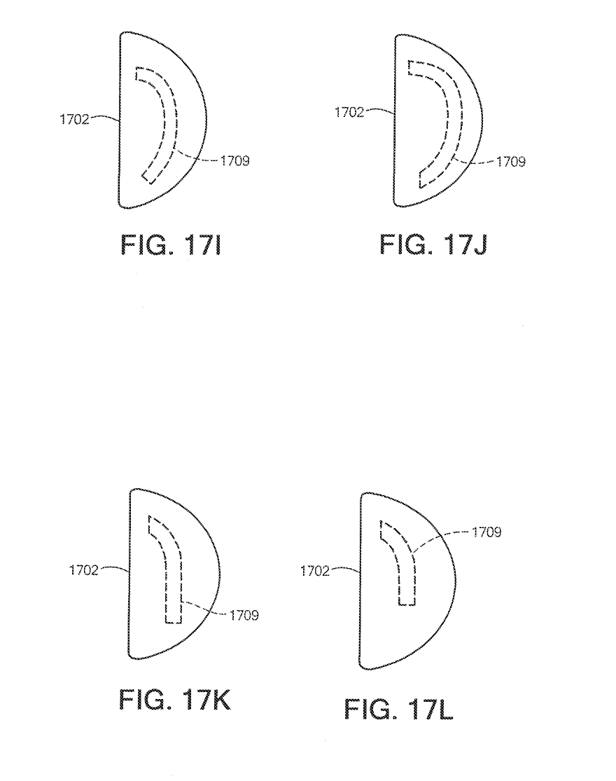

FIGS. 17A-D show joint implants 1700 wherein the bearing component 1701 is slideably engaged with the first component 1702, in accordance with various embodiments of the invention. The first surface 1706 of the bearing component 1701 includes an anchor 1708 (also referred to as an extender) that runs in a recessed slot 1709 in the first component 1702, thereby reducing the risk of dislocation of the top component relative to the bottom component.

FIGS. 17E-J show exemplary locations and configurations of the recessed slot 1709 of the first component 1702, in accordance with various embodiments of the invention. The shape of the recessed slot 1709 determines the direction in which the bearing component 1701 moves during gait or other knee activities. In FIG. 17E, only straight AP motion is possible. In FIG. 17F, the bearing component 1701 can move along a constant radius relative to the first component 1702. In FIG. 17G, the bearing component 1701 can move in anteroposterior direction; far anteriorly some rotation of the bearing component 1701 is enabled. In FIG. 17H, rotation of the bearing component 1701 will occur posteriorly, the recessed slot 1709 curved only posteriorly.

FIGS. 17I-J show embodiments where the radius of the recessed slot 1709 is variable in the axial plane thereby allowing not only AP movement, but also gradual, constant rotation of the bearing component 1701 with knee flexion and extension and gait.

FIG. 17K is an example of a joint device 1700 with a recessed slot 1704 allowing extensive AP motion and anteriorly also some rotation of the bearing component 1701. In FIG. 17L, the AP motion is restricted by shortening the length of the recessed slot 1704 in that dimension.

In further embodiments, the recessed slot 1704 may have a changing slope, further guiding the motion of the bearing component 1701.

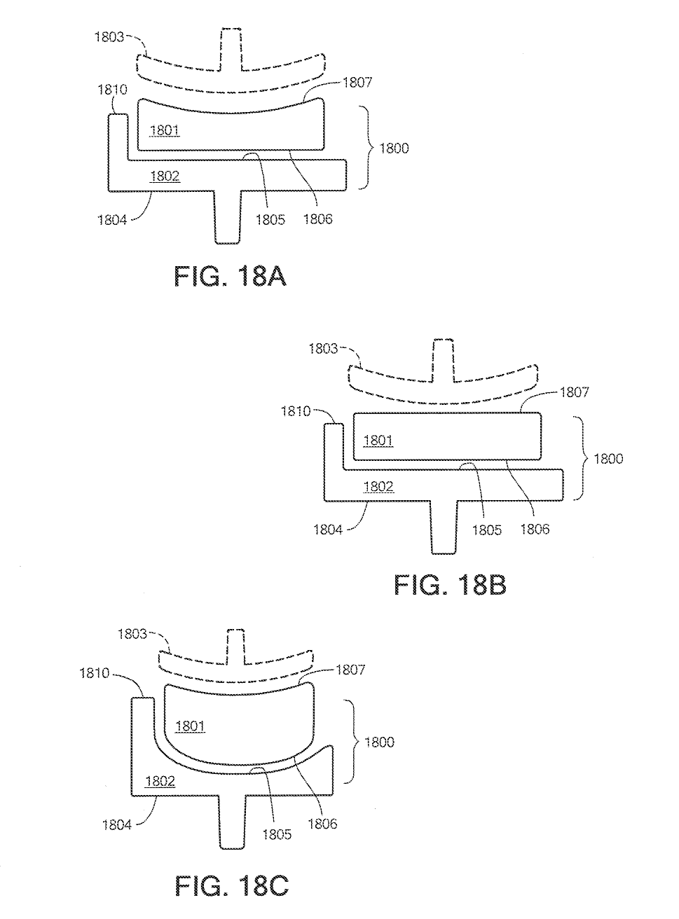

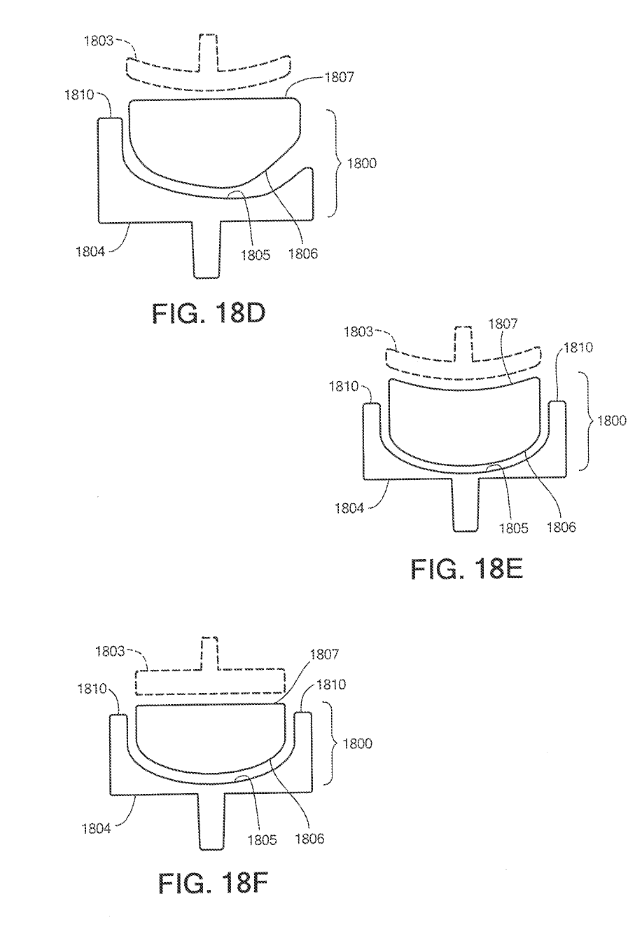

FIGS. 18A-F show a mobile bearing joint implant 1800 having a stop 1810 restricting motion of the bearing component 1801 in one or more dimensions, in accordance with one embodiment of the invention. There may be, without limitation, one stop (FIGS. 18A-D) or two stops (FIGS. 18E-F).

FIG. 18A shows a joint implant 1800 with a flat bearing surface in one or more dimensions between the bearing component 1801 and the first component 1802, a curvature on the second surface 1807 of the bearing component 1801 surface and a stop 1810 on the left restricting movement of the bearing component 1801 in this direction, in accordance with one embodiment of the invention. The stop 1810 is typically be located near the intercondylar notch, restricting movement of the bearing component 1801 in this direction. The stop, may be, without limitation, straight or curved. The stop may be sloped, allowing for increasing resistance, as opposed to an abrupt stop.

FIG. 18B shows a joint implant 1800 with a flat bearing surface in one or more dimensions between the bearing component 1801 and the first component 1802, a flat second surface 1807 of the bearing component 1801 surface facing the second component 1803 and a stop 1810 on the left restricting movement of the bearing component 1801 in this direction, in accordance with one embodiment of the invention.

FIG. 18C shows a joint implant 1800 with a curved bearing surface in one or more dimensions between the bearing component 1801 and the first component 1802, a curvature on the second surface 1807 of the bearing component 1801 surface and a stop 1810 on the left restricting movement of the bearing component 1801 in this direction, in accordance with one embodiment of the invention. The radius of the bearing surface between the bearing component 1801 and the first component 1802 may be substantially constant.

FIG. 18D shows a joint device 1800 with a curved bearing surface in one or more dimensions between the bearing component 1801 and the first component 1802, a flat second surface 1807 of the bearing component 1801 surface in one or more dimensions and a stop 1810 on the left restricting movement of the bearing component 1801 in this direction, in accordance with one embodiment of the invention. The radius of the bearing surface between the bearing component 1801 and the first component 1802 is variable. In this example, the radius of the external surface 1805 of the first component 1802 facing the bearing component 1801 is not only variable but also differs in some areas from the radius of first surface 1806 of the bearing component 1801 facing the first component 1802.

FIGS. 18E-F show joint implants 1800 with two stops 1810, e.g. one medial and one lateral or one anterior and one posterior, in accordance with various embodiments of the invention. In FIG. 18E, the bearing surface of the bearing and first component 1801 and 1802 is curved with a constant radius and the second surface 1807 of the bearing component 1801 facing, in a knee implant for example, the femur is curved with a substantially constant radius. In FIG. 18F, the bearing surface of the bearing and first component 1801 and 1802 is curved with a constant radius and the second surface 1807 of the bearing component 1801 facing, for example in a knee implant, the femur is fiat in one or more dimensions.

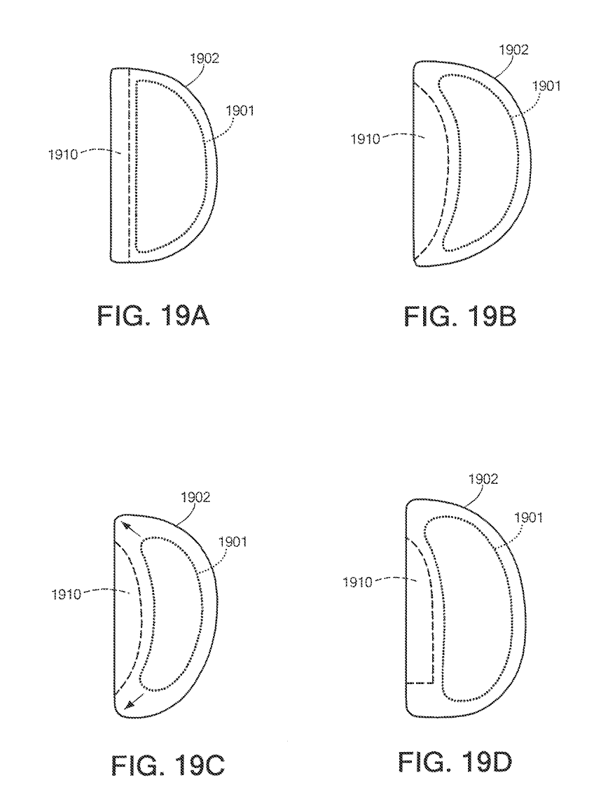

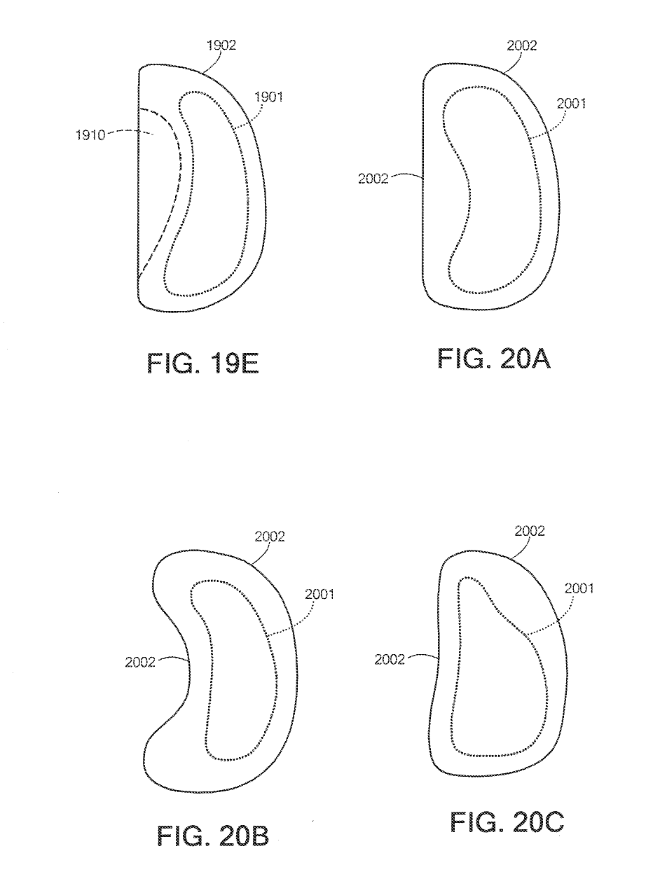

FIGS. 19A-E show various embodiments related to the stop 1910. The stop 1910 may be straight, for example oriented in anteroposterior direction (FIG. 19A). The stop 1910 may be curved with constant radius (FIG. 19B). The bearing component (stippled) 1901 may be large relative to the first component 1902, for example covering 85% of the first component 1902, thereby limiting the amount of anteroposterior movement and rotation (FIG. 19B). The bearing component (stippled) 1901 may be small relative to the first component 1902, for example covering only 70% of the first component 1902, thereby allowing for more anteroposterior movement and rotation (FIG. 19C).

The stop 1910 may be curved with variable radius and it can contain straight portions (FIG. 19D). The stop 1910 may be curved with variable radius (FIG. 19E), for example with a substantially matching bearing component 1901 (FIG. 19E). With this design, the stop 1910 can influence and guide the direction of the bearing component relative to the first component. Preferably, this guidance may be used to achieve near physiologic motion.

FIGS. 20A-C demonstrate top (stippled line) and bottom (solid line) bearing and first components 2001 and 2002, respectively, with different shapes, in accordance with various embodiments of the invention. The bearing component 2001 may substantially be the same or substantially differ from the shape of the first component 2002. Both bearing and first component 2001 and 2002 may have, without limitation, one or more straight, convex or concave portions.

The various joint implants described herein may be used, without limitation, in conjunction with knee implants, including a unicompartmental arthroplasty, medial or lateral; a bicompartmental arthroplasty that covers portions or all of one femoral condyle, medial or lateral, and the trochlea, and a total knee arthroplasty system. In a total knee arthroplasty system, the intercondylar region can be preserved by using a medial and a lateral tibial device in combination. Both devices may be a fixed, non-mobile bearing, both can be a mobile bearing, or one can be a fixed, non-mobile bearing, while the other is a mobile bearing. As described above, the joint implants described herein may also be implemented for the hip, ankle, shoulder, elbow, wrist, and hand.

In various embodiments, the joint implant may have one or more mobile bearings.

The various components used for the mobile bearing joint implant may be composed of metal, plastic, ceramic or any other material know in the art. Different components may be composed of different materials, e.g. one metal and one plastic. Alternatively, only the same material may be used for the bearing surfaces, e.g. ceramic. The bearing surfaces of each component may vary in material composition e.g. ceramic on the side facing the femoral condyle and metal on the undersurface.

As described herein, repair systems of various sizes, curvatures and thicknesses can be obtained. These repair systems can be catalogued and stored to create a library of systems from which an appropriate system for an individual patient can then be selected. In other words, a defect, or an articular surface, is assessed in a particular subject and a pre-existing repair system having a suitable shape and size is selected from the library for further manipulation (e.g., shaping) and implantation.

### IV. Manufacturing

### A. Shaping

In certain instances shaping of the repair material will be required before or after formation (e.g., growth to desired thickness), for example where the thickness of the required cartilage material is not uniform (e.g., where different sections of the cartilage replacement or regenerating material require different thicknesses).

The replacement material can be shaped by any suitable technique including, but not limited to, casting techniques, mechanical abrasion, laser abrasion or ablation, radiofrequency treatment, cryoablation, variations in exposure time and concentration of nutrients, enzymes or growth factors and any other means suitable for influencing or changing cartilage thickness. See, e.g., WO 00/15153 to Mansmann published Mar. 23, 2000; If enzymatic digestion is used, certain sections of the cartilage replacement or regenerating material can be exposed to higher doses of the enzyme or can be exposed longer as a means of achieving different thicknesses and curvatures of the cartilage replacement or regenerating material in different sections of said material.

The material can be shaped manually and/or automatically, for example using a device into which a pre-selected thickness and/or curvature has been input and then programming the device using the input information to achieve the desired shape.

In addition to, or instead of, shaping the cartilage repair material, the site of implantation (e.g., bone surface, any cartilage material remaining, etc.) can also be shaped by any suitable technique in order to enhance integration of the repair material.

### B. Sizing

The articular repair system can be formed or selected so that it will achieve a near anatomic fit or match with the surrounding or adjacent cartilage, subchondral bone, menisci and/or other tissue. The shape of the repair system can be based on the analysis of an electronic image (e.g. MRI, CT, digital tomosynthesis, optical coherence tomography or the like). If the articular repair system is intended to replace an area of diseased cartilage or lost cartilage, the near anatomic fit can be achieved using a method that provides a virtual reconstruction of the shape of healthy cartilage in an electronic image.

In one embodiment of the invention, a near normal cartilage surface at the position of the cartilage defect can be reconstructed by interpolating the healthy cartilage surface across the cartilage defect or area of diseased cartilage. This can, for example, be achieved by describing the healthy cartilage by means of a parametric surface (e.g. a B-spline surface), for which the control points are placed such that the parametric surface follows the contour of the healthy cartilage and bridges the cartilage defect or area of diseased cartilage. The continuity properties of the parametric surface will provide a smooth integration of the part that bridges the cartilage defect or area of diseased cartilage with the contour of the surrounding healthy cartilage. The part of the parametric surface over the area of the cartilage defect or area of diseased cartilage can be used to determine the shape or part of the shape of the articular repair system to match with the surrounding cartilage.

In another embodiment, a near normal cartilage surface at the position of the cartilage defect or area of diseased cartilage can be reconstructed using morphological image processing. In a first step, the cartilage can be extracted from the electronic image using manual, semi-automated and/or automated segmentation techniques (e.g., manual tracing, region growing, live wire, model-based segmentation), resulting in a binary image. Defects in the cartilage appear as indentations that can be filled with a morphological closing operation performed in 2-D or 3-D with an appropriately selected structuring element. The closing operation is typically defined as a dilation followed by an erosion. A dilation operator sets the current pixel in the output image to 1 if at least one pixel of the structuring element lies inside a region in the source image. An erosion operator sets the current pixel in the output image to 1 if the whole structuring element lies inside a region in the source image. The filling of the cartilage defect or area of diseased cartilage creates a new surface over the area of the cartilage defect or area of diseased cartilage that can be used to determine the shape or part of the shape of the articular repair system to match with the surrounding cartilage or subchondral bone.

As described above, the articular repair system can be formed or selected from a library or database of systems of various sizes, curvatures and thicknesses so that it will achieve a near anatomic fit or match with the surrounding or adjacent cartilage and/or subchondral bone. These systems can be pre-made or made to order for an individual patient. In order to control the fit or match of the articular repair system with the surrounding or adjacent cartilage or subchondral bone or menisci and other tissues preoperatively, a software program can be used that projects the articular repair system over the anatomic position where it will be implanted. Suitable software is commercially available and/or readily modified or designed by a skilled programmer.

In yet another embodiment, the articular surface repair system can be projected over the implantation site using one or more 3-D images. The cartilage and/or subchondral bone and other anatomic structures are extracted from a 3-D electronic image such as an MRI or a CT using manual, semi-automated and/or automated segmentation techniques. A 3-D representation of the cartilage and/or subchondral bone and other anatomic structures as well as the articular repair system is generated, for example using a polygon or NURBS surface or other parametric surface representation. For a description of various parametric surface representations see, for example Foley, J. D. et al., Computer Graphics: Principles and Practice in C; Addison-Wesley, 2^{nd} edition, 1995).

The 3-D representations of the cartilage and/or subchondral bone and other anatomic structures and the articular repair system can be merged into a common coordinate system. The articular repair system can then be placed at the desired implantation site. The representations of the cartilage, subchondral bone, menisci and other anatomic structures and the articular repair system are rendered into a 3-D image, for example application programming interfaces (APIs) OpenGL® (standard library of advanced 3-D graphics functions developed by SGI, Inc.; available as part of the drivers for PC-based video cards, for example from www.nvidia.com for NVIDIA video cards or www.3dlabs.com for 3Dlabs products, or as part of the system software for Unix workstations) or DirectX® (multimedia API for Microsoft Windows® based PC systems; available from www.microsoft.com). The 3-D image can be rendered showing the cartilage, subchondral bone, menisci or other anatomic objects, and the articular repair system from varying angles, e.g. by rotating or moving them interactively or non-interactively, in real-time or non-real-time.

The software can be designed so that the articular repair system, including surgical tools and instruments with the best fit relative to the cartilage and/or subchondral bone is automatically selected, for example using some of the techniques described above. Alternatively, the operator can select an articular repair system, including surgical tools and instruments and project it or drag it onto the implantation site using suitable tools and techniques. The operator can move and rotate the articular repair systems in three dimensions relative to the implantation site and can perform a visual inspection of the fit between the articular repair system and the implantation site. The visual inspection can be computer assisted. The procedure can be repeated until a satisfactory fit has been achieved. The procedure can be performed manually by the operator; or it can be computer-assisted in whole or part. For example, the software can select a first trial implant that the operator can test. The operator can evaluate the fit. The software can be designed and used to highlight areas of poor alignment between the implant and the surrounding cartilage or subchondral bone or menisci or other tissues. Based on this information, the software or the operator can then select another implant and test its alignment. One of skill in the art will readily be able to select, modify and/or create suitable computer programs for the purposes described herein.

In another embodiment, the implantation site can be visualized using one or more cross-sectional 2-D images. Typically, a series of 2-D cross-sectional images will be used. The 2-D images can be generated with imaging tests such as CT, MRI, digital tomosynthesis, ultrasound, or optical coherence tomography using methods and tools known to those of skill in the art. The articular repair system can then be superimposed onto one or more of these 2-D images. The 2-D cross-sectional images can be reconstructed in other planes, e.g. from sagittal to coronal, etc. Isotropic data sets (e.g., data sets where the slice thickness is the same or nearly the same as the in-plane resolution) or near isotropic data sets can also be used. Multiple planes can be displayed simultaneously, for example using a split screen display. The operator can also scroll through the 2-D images in any desired orientation in real time or near real time; the operator can rotate the imaged tissue volume while doing this. The articular repair system can be displayed in cross-section utilizing different display planes, e.g. sagittal, coronal or axial, typically matching those of the 2-D images demonstrating the cartilage, subchondral bone, menisci or other tissue. Alternatively, a three-dimensional display can be used for the articular repair system. The 2-D electronic image and the 2-D or 3-D representation of the articular repair system can be merged into a common coordinate system. The articular repair system can then be placed at the desired implantation site. The series of 2-D cross-sections of the anatomic structures, the implantation site and the articular repair system can be displayed interactively (e.g. the operator can scroll through a series of slices) or non-interactively (e.g. as an animation that moves through the series of slices), in real-time or non-real-time.

### C. Rapid Prototyping

Rapid prototyping is a technique for fabricating a three-dimensional object from a computer model of the object. A special printer is used to fabricate the prototype from a plurality of two-dimensional layers. Computer software sections the representations of the object into a plurality of distinct two-dimensional layers and then a three-dimensional printer fabricates a layer of material for each layer sectioned by the software. Together the various fabricated layers form the desired prototype. More information about rapid prototyping techniques is available in US Patent Publication No 2002/0079601A1 to Russell et al., published Jun. 27, 2002. An advantage to using rapid prototyping is that it enables the use of free form fabrication techniques that use toxic or potent compounds safely. These compounds can be safely incorporated in an excipient envelope, which reduces worker exposure

A powder piston and build bed are provided. Powder includes any material (metal, plastic, etc.) that can be made into a powder or bonded with a liquid. The power is rolled from a feeder source with a spreader onto a surface of a bed. The thickness of the layer is controlled by the computer. The print head then deposits a binder fluid onto the powder layer at a location where it is desired that the powder bind. Powder is again rolled into the build bed and the process is repeated, with the binding fluid deposition being controlled at each layer to correspond to the three-dimensional location of the device formation. For a further discussion of this process see, for example, US Patent Publication No 2003/017365A1 to Monkhouse et al. published Sep. 18, 2003.

The rapid prototyping can use the two dimensional images obtained, as described above in Section I, to determine each of the two-dimensional shapes for each of the layers of the prototyping machine. In this scenario, each two dimensional image slice would correspond to a two dimensional prototype slide. Alternatively, the three-dimensional shape of the defect can be determined, as described above, and then broken down into two dimensional slices for the rapid prototyping process. The advantage of using the three-dimensional model is that the two-dimensional slices used for the rapid prototyping machine can be along the same plane as the two-dimensional images taken or along a different plane altogether.

Rapid prototyping can be combined or used in conjunction with casting techniques. For example, a shell or container with inner dimensions corresponding to an articular repair system can be made using rapid prototyping. Plastic or wax-like materials are typically used for this purpose. The inside of the container can subsequently be coated, for example with a ceramic, for subsequent casting. Using this process, personalized casts can be generated.

Rapid prototyping can be used for producing articular repair systems. Rapid prototyping can be performed at a manufacturing facility. Alternatively, it may be performed in the operating room after an intraoperative measurement has been performed.

### V. Surgical Techniques

Prior to performing surgery on a patient, the surgeon can preoperatively make a determination of the alignment of the knee using, for example, an erect AP x-ray. In performing preoperative assessment any lateral and patella spurs that are present can be identified.

Using standard surgical techniques, the patient is anesthetized and an incision is made in order to provide access to the portion or portions of the knee joint to be repaired. A medial portal can be used initially to enable arthroscopy of the joint. Thereafter, the medial portal can be incorporated into the operative incision and/or standard lateral portals can be used.

Once an appropriate incision has been made, the exposed compartment is inspected for integrity, including the integrity of the ligament structures. If necessary, portions of the meniscus can be removed as well as any spurs or osteophytes that were identified in the AP x-ray or that may be present within the joint. In order to facilitate removal of osteophytes, the surgeon may flex the knee to gain exposure to additional medial and medial-posterior osteophytes. Additionally, osteophytes can be removed from the patella during this process. If necessary, the medial and/or lateral meniscus can also be removed at this point, if desired, along with the rim of the meniscus.

As would be appreciated by those of skill in the art. evaluation of the medial cruciate ligament may be required to facilitate tibial osteophyte removal.

Once the joint surfaces have been prepared, the desired implants can be inserted into the joint.

### A. Tibial Plateau

To insert the device 200 of FIG. 2 into the medial compartment, perform a mini-incision arthrotomy medial to the patella tendon. Once the incision is made, expose the medial condyle and prepare a medial sleeve to about 1 cm below the joint line using a suitable knife and curved osteotome. After preparing the medial sleeve, place a Z-retractor around the medial tibial plateau and remove anterior portions of the meniscus and the osteophytes along the tibia and femur. At this point, the knee should be flexed to about 60° or more. Remove the Z-retractor and place the implant against the most distal aspect of the femur and over the tibial plateau edge. Push the implant straight back. In some instances, application of valgus stress may ease insertion of the implant.

To insert the device of FIG. 2 into the lateral compartment, perform a mini-incision arthrotomy lateral to the patella tendon. Once the incision is made, expose the lateral condyle and prepare a lateral sleeve to about 1 cm below the joint line using a suitable knife and curved osteotome. After preparing the lateral sleeve, place a Z-retractor around the lateral tibial plateau and remove anterior portions of the meniscus and the osteophytes along the tibia and femur. Remove the Z-retractor and place the implant against the distal aspect of the femur and over the tibial plateau edge. Hold the implant at a 45° angle and rotate the implant against the lateral condyle using a lateral to medial push toward the lateral spine. In some instances, application of varus stress may ease insertion of the implant.

Once any implant shown in FIG. 2 is implanted, the device should be positioned within 0 to 2 mm of the AP boundaries of the tibial plateau and superimposed over the boundary. Verification of the range of motion should then be performed to confirm that there is minimal translation of the implant. Once positioning is confirmed, closure of the wound is performed using techniques known in the art.

As will be appreciated by those of skill in the art, additional treatment of the surface of the tibial plateau may be desirable depending on the configuration of the implant 200. For example, one or more channels or grooves may be formed on the surface of the tibial plateau to accommodate anchoring mechanisms such as the keel 212 shown in FIG. 2K or the translational movement cross-members 222, 221 shown in FIGS. 2M-N.

### B. Condylar Repair Systems

To insert the device 300 shown in FIG. 3, depending on the condyle to be repaired either an antero-medial or antero-lateral skin incisions is made which begins approximately 1 cm proximal to the superior border of the patella. The incision typically can range from, for example, 6-10 cm along the edge of the patella. As will be appreciated by those of skill in the art, a longer incision may be required under some circumstances.

It may be required to excise excess deep synovium to improve access to the joint. Additionally, all or part of the fat pad may also be excused and to enable inspection of the opposite joint compartment.

Typically, osteophytes are removed from the entire medial and/or lateral edge of the femur and the tibia as well as any osteophytes on the edge of the patella that might be significant.

Although it is possible, typically the devices 300 do not require resection of the distal femur prior to implanting the device. However, if desired, bone cuts can be performed to provide a surface for the implant.

At this juncture, the patient's leg is placed in 90° flexion position. I guide can then be placed on the condyle which covers the distal femoral cartilage. The guide enables the surgeon to determine placement of apertures that enable the implant 300 to be accurately placed on the condyle. With the guide in place, holes are drilled into the condyle to create apertures from 1-3 mm in depth. Once the apertures have been created, the guide is removed and the implant 300 is installed on the surface of the condyle. Cement can be used to facilitate adherence of the implant 300 to the condyle.

Where more than one condyle is to be repaired, e.g., using two implants 300 of FIG. 3, or the implant 400 of FIG. 4, or where a condyle and a portion of the patellar surface is to be repaired, e.g., using the implant 500 of FIG. 5, the surgical technique described herein would be modified to, for example, provide a greater incision for accessing the joint, provide additional apertures for receiving the pegs of the implant, etc.

### C. Patellar Repair System

To insert the device shown in FIG. 7, it may be appropriate to use the incisions made laterally or medially to the patella tendon and described above with respect to FIG. 2. First the patella is everted laterally and the fat pad and synovium are bent back from around the periphery of the patella. If desired, osteophytes can be removed. Prior to resurfacing the natural patella 620, the knee should be manually taken through several range of motion maneuvers to determine whether subluxation is present. If subluxation is present, then it may be necessary to medialize the implant 600. The natural patella can then be cut in a planar, or flat, manner such that a flat surface is presented to the implant. The geometric center of the patella 620 is then typically aligned with the geometric center of the implant 600. In order to anchor the implant 600 to the patella 620, one or more holes or apertures 612 can be created in the patellar surface to accept the pegs 610 of the implant 600.

### VI. Kits

One or more of the implants described above can be combined together in a kit such that the surgeon can select one or more implants to be used during surgery.

The foregoing description of embodiments of the present invention has been provided for the purposes of illustration and description. It is not intended to be exhaustive or to limit the invention to the precise forms disclosed. Many modifications and variations will be apparent to the practitioner skilled in the art. The embodiments were chosen and described in order to best explain the principles of the invention and its practical application, thereby enabling others skilled in the art to understand the invention and the various embodiments and with various modifications that are suited to the particular use contemplated.

What is claimed is:

1.-30. (canceled)

31. An implant for bone re-surfacing in a joint, the implant comprising:
a bearing platform having a front surface which forms a bearing surface and a back surface; and
securing means projecting from the back surface, the securing means having a locking surface arranged to bear against an undercut surface of the bone to secure the implant against the bone.

32. An implant according to claim 31, wherein said locking surface is angled at least partly towards the back surface of the bearing platform so that a space is defined between the locking surface and the back surface into which a portion of the bone can extend to secure the implant against the bone.

33. An implant according to claim 31, wherein the locking surface extends in an insertion direction in which the implant can be moved to insert the implant.

34. An implant according to claim 31, wherein the securing means is in the form of a rib extending along the back of the bearing platform.

35. An implant according to claim 34, wherein one side of the rib is undercut so as to form an overhang so that a portion of the bone can project under the overhang so as to secure the implant to the bone.

36. An implant according to claim 31, wherein the securing means is straight so that the implant can be inserted in a straight line to secure it to the bone.

37. An implant according to claim 31, wherein the securing means is curved so that the implant can be inserted along a curved path to secure it to the bone.

38. An implant according to claim 31, wherein the securing means is one of a pair of securing means extending parallel to each other along the implant.

39. An implant according to claim 31, additionally comprising a tool engaging formation arranged to engage an insertion tool.

40. An implant according to claim 31, wherein the locking surface is angled relative to the back surface of the bearing platform so as to urge the back surface against the bone as the implant is inserted.

41. An implant according to claim 31, wherein said implant is a tibial implant arranged to resurface one of the medial and lateral tibial plateaux.

42. An implant according to claim 41, wherein the bearing platform has an anterior portion the upper surface of which is angled downwards relative to the bearing surface.

43. An implant according to claim 41, wherein the bearing platform has an anterior portion the back surface of which is angled downwards so as to abut against the bone when the implant is fully inserted.

44. An implant according to claim 41, wherein the bearing platform has an abutment edge at its posterior end arranged to abut against the edge of a recess in the tibia when the implant is fully inserted.

45. An implant according to claim 31, wherein at least a part of the back surface of the bearing platform is convex in a coronal plane.

46. An implant according to claim 31, wherein at least a part of the bearing surface is concave in a coronal plane.

47. An implant according to claim 31, wherein at least a part of the bearing surface is convex in a sagittal plane.

48. An implant according to claim 31, wherein at least a part of the bearing surface is concave in the sagittal plane.

49. An implant according to claim 31, wherein the bearing surface comprises an anterior area and a posterior area, the two areas having different radii of curvature.

50. An implant according to claim 49, wherein the radius of curvature of the anterior area is greater than the radius of curvature of the posterior area.

51. An implant according to claim 49, wherein the bearing surface includes a transition region between the anterior and posterior areas.

52. An implant according to claim 50, wherein the transition region takes up no more than 10% of the length of the bearing surface in the sagittal plane.

53. An implant according to claim 31, wherein the bearing surface and the back surface of the bearing platform are both curved so that the majority of the bearing platform is of a substantially constant thickness.

54. A uni-condylar implant set comprising:
a tibial implant comprising a bearing platform having a front surface which forms a bearing surface and a back surface, and securing means projecting from the back surface, the securing means having a locking surface arranged to bear against an undercut surface of the bone to secure the implant against the bone; and
a femoral implant.

55. An implant set according to claim 54, wherein the femoral implant comprises a femoral bearing portion having a bearing surface which is convex on a sagittal plane, the bearing surface having an anterior area and a posterior area, the areas having different radii of curvature.

56. An implant set according to claim 55, wherein the anterior area has a greater radius of curvature than the posterior area.

57. An implant set according to claim 55, wherein the bearing surface includes a transition region between the anterior and posterior areas.

58. An implant according to claim 57, wherein the transition region takes up no more than 10% of the length of the bearing surface in the sagittal plane.

59. An implant set according to claim 54, wherein the bearing surface of the femoral component is arranged to contact the bearing surface of the tibial component.

60. An implant set according to claim 54, further comprising a meniscal component arranged to be located between the tibial and femoral components and having upper and lower bearing surfaces arranged to contact the femoral and tibial components respectively.

61. An implant set according to claim 60, wherein the lower bearing surface of the meniscal component is convex in both a coronal plane and a sagittal plane.

62. An implant set according to claim 60, wherein the lower bearing surface of the meniscal component is convex in the coronal plane and concave in the sagittal plane.

63. An implant set according to claim 60, wherein the upper bearing surface of the meniscal component is concave in both a coronal plane and a sagittal plane.
